(19) United States
(12) Patent Application Publication
(10) Pub. No.: US 2005/0276779 A1
(43) Pub. Date: Dec. 15, 2005

(54) Two coat cosmetic product, its method of use and make-up kit comprising this product

(75) Inventor: Xavier Blin, Paris (FR)

Correspondence Address: FINNEGAN, HENDERSON, FARABOW, GARRETT & DUNNER;LLP, 901 NEW YORK AVENUE, NW, WASHINGTON, DC, 20001-4413, US

(73) Assignee: L'OREAL

(21) Appl. No.: 11/136,468

(22) Filed: May 25, 2005

Related U.S. Application Data
(60) Provisional application No. 60/575,800, filed on Jun. 2, 2004.

(30) Foreign Application Priority Data
May 25, 2004 (FR) 04 51026

(51) Int. Cl. A61K 7/06; A61K 7/11

(52) U.S. Cl. 424/70.16

(57) ABSTRACT

The present disclosure relates to a cosmetic product comprising at least one first and at least one second compositions, wherein the at least one first composition comprises a dispersion, in a liquid fatty phase, of particles of at least one graft ethylene polymer and the at least one second composition comprises a cosmetically acceptable medium. The present disclosure also relates to a make-up method and to a make-up kit comprising the cosmetic product.

This application claims benefit of U.S. Provisional Application No. 60/575,800, filed Jun. 2, 2004, the contents of which are incorporated herein by reference. This application also claims benefit of priority under 35 U.S.C. § 119 to French Patent Application No. 04 51026, filed May 25, 2004, the contents of which are also incorporated by reference.

The present disclosure relates to a cosmetic product comprising at least two compositions which may be applied successively to the skin both of the face and of the body, to the lower and upper eyelids, to the lips and to superficial body growths such as the nails, the eyebrows, the eyelashes and the hair. The present disclosure also relates to a method for making up the face and the body using these two compositions.

Each composition of the at least two compositions as disclosed herein may be a foundation, a make-up rouge, an eyeshadow, a concealer product, a blusher, a loose or compacted powder, a lipstick, a lip balm, a lip gloss, a lip or eye pencil, a mascara, an eye liner, a nail varnish or a make-up product for the body or a coloring product for the skin.

Thus it would be desirable to provide a cosmetic product that exhibits good staying-power, is glossy, and is endowed with transfer-resistant and gloss properties. The present inventor has found that the cosmetic products disclosed herein have at least one of these properties. For example, the cosmetic product according to the present disclosure can have good staying power, and can be transfer-resistant and glossy.

A poor staying power over time may result in, for instance, a poor staying power of the color and/or in a poor staying power of the gloss over time. This poor staying power may be characterized by a modification of the color (color change, fading) generally following an interaction with the sebum and/or the sweat secreted by the skin in the case of a foundation or a blusher, or an interaction with the saliva in the case of lipsticks; or may be characterized by a reduction in the gloss, in the case of a nail varnish. This poor staying power can require the user to reapply the make-up very often, which may constitute a loss of time.

The so-called “transfer-resistant” make-up compositions for the lips and the skin are compositions which have the advantage of forming a deposit which does not become deposited, at least in part, on the supports with which they are brought into contact (glass, clothing, cigarette, fabric).

The known transfer-resistant compositions are generally based on silicone resins and volatile silicone oils, and, although having improved staying power properties, they may have the disadvantage of leaving on the skin and the lips, after evaporation of the volatile silicone oils, a film which becomes uncomfortable over time (e.g., the sensation of dryness and tightness), which can turn the user off to this type of lipstick.

In addition, these compositions based on volatile silicone oils and silicone resins lead to films having a matte color. Nowadays, consumers are more in search of products, for example, for coloring the lips or the eyelids, which are glossy while at the same time have good staying power and/or are transfer-resistant.

To obtain products with good staying power and which are transfer-resistant, use is often made of volatile oils or polymers in dispersion in a volatile solvent. However, these products may not be sufficiently glossy. A cosmetic product has been envisaged which comprises two compositions, a first comprising a polymer in dispersion in a volatile solvent, over which is applied a second glossy and greasy composition (FR-A-2823101). The staying power of the color of the make-up obtained with these two compositions is good, but the disappearance of the second composition in the course of the day when it is worn can result in a loss of gloss, and the gloss is desired by the consumer.

The company Kose has moreover proposed, in the Japanese Patent Application No. JP-A-0 5 221 829, the use of a gel based on perfluorinated materials, which is applied over a film of lipstick so as to prevent it from being transferred onto other surfaces, the gel being incompatible with the film of the lipstick.

There may also be mentioned International Patent Application No. WO-A-97/17057, which describes a method for increasing the staying power and transfer-resistance properties, comprising applying two compositions one over the other. The composition to be applied has an overall Hildebrand solubility parameter of less than 8.5 (cal/cm³)^{1/2}, and the composition to be applied as top coat should contain oils whose calculated partition coefficient ClogP is at least equal to 13.

U.S. Pat. No. 6,001,374 proposes a multicoat make-up system which comprising using a composition containing a resin soluble in alcohol and insoluble in water, which may be applied as base coat or as top coat, and which can have the advantage of not staining an object brought into contact with the make-up and of being resistant to water and to rubbing, while having a certain level of gloss. However, this composition contains a water-soluble alcohol, in particular ethanol, a compound which can have an irritant, dehydrating character for the skin and more especially for the lips, and which may be particularly uncomfortable when the skin or the lips are damaged.

In International Patent Application No. WO 02/067877, a method for improving the aesthetic properties of a transfer-resistant composition, comprising applying a second composition over a film of transfer-resistant composition, is described. The second composition disclosed therein must not chemically interact with the transfer-resistant composition so as not to adversely affect its cosmetic properties. Some products described in this document can have an unpleasant odor and are sticky. Other products may not be sufficiently glossy.

Accordingly, the present disclosure relates to a novel route for formulating a cosmetic product, for instance a make-up product, of the type comprising at least two compositions to be applied successively one after the other. This cosmetic product may advantageously exhibit good gloss, transfer-resistance and/or staying power properties.

One aspect of the present disclosure is, for example, to produce such a cosmetic product endowed with good transfer-resistance and gloss staying power properties.

Another aspect of the present disclosure is also to provide a cosmetic product, such as a make-up product, which may exhibit at least one property chosen from the properties of “transfer-resistance,” non-migration, staying power of the color, comfort, non-dehydration, gloss and staying power of the gloss over time.

The inventor has observed, surprisingly, that these aspects could be achieved by combining at least one first composition comprising a dispersion, in a liquid fatty phase, of particles of a graft ethylene polymer, and at least one second composition comprising a cosmetically acceptable medium. The at least one second composition can comprise, for instance, at least one oil.

Thus, the compositions according to at least some of the aspects of the present disclosure can make it possible to obtain a cosmetic result that is very glossy on application and over time, which does not migrate, does not transfer, has good staying power, while being comfortable on application and over time (i.e., non-sticky, non-dehydrating, no tightness).

For example, the product of the present disclosure can make it possible to obtain non-sticky continuous deposits on the skin or the lips, with good coverage, having a very glossy appearance, which can be adapted to the desire of the consumer, may not migrate, may not transfer and can have good staying power, may not be oily, may not dry out the skin, the hair or the lips to which it is applied, both during application and over time. It can have, in addition, good stability properties and thus may allow homogeneous and aesthetic application.

It has also been observed that compositions of the product according to the present disclosure can have advantageous qualities of spreading and adhesion onto the skin, the lips or the eyelashes, as well as an unctuous and pleasant feel.

The properties of staying power, transfer-resistance and non-migration, which can be combined with the glossy and non greasy appearance, make a product that is beneficial for producing make-up products for the lips such as lipsticks and lip glosses, for the eyes such as mascara, eye liners and eyeshadows, and for the nails or for the hair.

One aspect of the present disclosure is therefore a cosmetic product intended to be applied to the skin, such as the skin of the face or the neck, the lips or the eyelids, comprising at least one first and at least one second composition, wherein the at least one first composition comprises a dispersion, in a liquid fatty phase, of particles of a graft ethylene polymer, and the at least one second composition, which is different from the first, comprises a cosmetically acceptable medium.

Another aspect of the present disclosure is a cosmetic product intended to be applied to the superficial body growths, such as the nails, the hair or the eyebrows, comprising at least one first and at least one second compositions, wherein the at least one first composition comprises, in a cosmetically acceptable organic liquid medium, at least one graft ethylene polymer, and the at least one second composition, which is different from the first, comprises a cosmetically acceptable medium.

The product of the present disclosure can be, for instance, a make-up product for the skin, the nails or the hair.

As used herein, the term “make-up product” is understood to mean a product comprising at least one coloring agent allowing the deposition of a color onto a keratin material (such as the skin or the superficial body growths) of a human being by applying onto the keratin material, products such as lipsticks, blushers, eye liners, foundations, self-tanning agents, semi-permanent make-up products (tattoo) and the like.

The product according to the present disclosure comprises at least two cosmetically acceptable compositions packaged separately or together in the same packaging item or in at least two separate packaging items.

For example, in one embodiment of the present disclosure, the at least one first and at least one second compositions are packaged separately, such as in separate packaging items.

One aspect of the present disclosure therefore, for example, is a make-up cosmetic product provided in the form of a lipstick, a foundation, a mascara, a blusher or an eye shadow, a lipstick, a nail varnish, a product having, for instance, care properties, a mascara, an eye liner, a concealer product or a make-up product for the body (of the tattoo type) or the hair.

Another aspect of the present disclosure is also a make-up kit comprising a make-up cosmetic product as defined above, wherein the at least one first and at least one second compositions are packaged separately and may be, for example, accompanied by appropriate application tools. These tools may be fine brushes, coarse brushes, pens, pencils, felts, quills, sponges, tubes and/or foam tips.

The at least one first composition of the product according to the present disclosure may act as a base coat applied to the keratin material and the at least one second composition may act as a top coat. It is, however, possible to apply under the at least one first composition an undercoat which may or may not have the constitution of the at least one second composition.

It is also possible to deposit an overcoat on the at least one second composition that may or may not have an identical constitution to that of the at least one first composition. In one embodiment of the present disclosure, the make-up product obtained is a two-coat make-up.

The at least one second composition may also act as a base coat applied to the keratin material and the at least one first composition may act as a top coat.

For example, the base coat can be a lipstick, a foundation, a mascara, a lip gloss, an eye liner, a nail varnish, a care product for the nails, and a make-up product for the body, and the top coat can be a care or protective product.

The present disclosure also relates to a method for making up the skin and/or the lips and/or the superficial body growths, which comprises applying a cosmetic product as defined above to the skin and/or the lips and/or the superficial body growths.

Another aspect of the present disclosure is also a cosmetic care or make-up method for the skin and/or the lips and/or the superficial body growths of a human being, which comprises applying to the skin, the lips and/or the superficial body growths a first coat of the at least one first composition comprising a dispersion, in a liquid fatty phase, of particles of a graft ethylene polymer, and then in applying, over all or part of the first coat, a second coat of the at least one second composition comprising a cosmetically acceptable medium. This method may comprise applying to the skin, the lips and/or the superficial body growths of a human being a first coat of the at least one first composition comprising a dispersion, in a liquid fatty phase, of particles of a graft ethylene polymer, in leaving the first coat to dry, and then in applying, over all or part of the first coat, a second coat of the at least one second composition comprising a cosmetically acceptable medium.

This method may also comprise applying to the skin, the lips and/or the superficial body growths of a human being a first coat of the at least one second composition comprising a cosmetically acceptable medium, in leaving the said first coat to dry, and then in applying, over all or part of the first coat, a second coat of the at least one first composition comprising a dispersion, in a liquid fatty phase, of particles of a graft ethylene polymer.

The product according to the present disclosure can be applied to the skin of the face, the scalp, the body, the lips, the inside of the lower eyelids and the superficial body growths such as the nails, the eyelashes, the hair, the eyebrows or even body hair. The at least one second composition may form units and may be applied with a pen, a pencil or any other applicator (sponge, finger, fine brush, coarse brush, quill). The make-up product as disclosed herein may also be applied to make-up accessories such as false nails, false eyelashes, wigs or pastilles or patches that adhere to the skin or the lips (of the beauty spot type).

One aspect of the present disclosure is also a made-up support comprising a first coat of the at least one first composition comprising a dispersion, in a liquid fatty phase, of particles of a graft ethylene polymer, and a second coat of the at least one second composition comprising a cosmetically acceptable medium, wherein the first coat is applied to the support first or over all or part of the second coat. The support may be, for instance, a hairpiece such as a wig, false nails, false eyelashes or patches that adhere to the skin or the lips (of the beauty spot type).

The present disclosure also relates to the cosmetic use of the cosmetic product defined above to improve the properties of comfort, such as the properties of non-stickiness and/or of non-dehydration, and/or of gloss and/or of transfer and/or of migration and/or of staying power of the make-up on the skin and/or the lips and/or the superficial body growths.

The present disclosure further relates to the use of a cosmetic product comprising at least one first and at least one second compositions, wherein the at least one first composition comprises a dispersion, in a liquid fatty phase, of particles of a graft ethylene polymer as described below, and the at least one second composition comprises a cosmetically acceptable medium, to confer on the skin and/or the lips and/or the superficial body growths a comfortable and/or glossy and/or transfer-resistant and/or non-migrating and/or good staying power cosmetic result.

The At Least One First Composition

The at least one first composition, according to the present disclosure, comprises a dispersion of particles, such as solid particles, of a graft ethylene polymer in a liquid fatty phase.

As used herein, the expression “ethylene” polymer is understood to mean a polymer obtained by polymerization of monomers comprising an ethylenic unsaturation.

The graft ethylene polymer dispersion can be, for example, free of a stabilizing polymer which is different from the graft polymer, such as those described in European Patent No. EP 749747, and the graft ethylene polymer particles would therefore not be surface-stabilized by such additional stabilizing polymers. The graft polymer disclosed herein may therefore be dispersed in the liquid fatty phase in the absence of an additional stabilizer at the surface of the graft polymer particles.

As used herein, the term “graft polymer” is understood to mean a polymer having a backbone-comprising at least one pendant side chain or situated at the chain end. In one embodiment, the side chain is pendant.

For example, the graft ethylene polymer may comprise an ethylene backbone that is insoluble in the liquid fatty phase, and side chains that are covalently linked to the backbone and that are soluble in the liquid fatty phase.

The graft ethylene polymer can be, for instance, a noncrosslinked polymer. For example, the polymer can be obtained by polymerization of monomers comprising a single polymerizable group.

In one embodiment of the present disclosure, for example, the graft ethylene polymer is a film-forming polymer.

As used herein, the term “film-forming” polymer is understood to mean a polymer capable of forming on its own, or in the presence of a film-forming aid, a continuous and adherent film on a support, such as on keratin materials, for instance, a cohesive film, such as a film whose cohesion and mechanical properties are such that the said film can be isolated from the support.

According to one embodiment of the present disclosure, the graft ethylene polymer is a graft acrylic polymer.

The graft ethylene polymer may be obtained, for example, by free-radical polymerization in an organic medium for polymerization:
of at least one ethylene monomer, such as of at least one acrylic monomer and optionally of at least one additional nonacrylic vinyl monomer, to form the insoluble backbone; and
of at least one macromonomer containing a polymerizable terminal group, to form the side chains, wherein the macromonomer has a weight-average molecular mass greater than or equal to 200 and the polymerized macromonomer is present in an amount ranging from 0.05% to 20% by weight, relative to the weight of the polymer.

The liquid fatty phase may comprise the organic polymerization medium.

The organic liquid dispersion medium, corresponding to the medium in which the graft polymer is provided, may be identical to the polymerization medium.

However, the polymerization medium may be replaced completely or partially by another organic liquid medium. This other organic liquid medium may be added, after polymerization, to the polymerization medium. The polymerization medium is then evaporated completely or partially.

The liquid fatty phase may comprise at least one organic liquid compound other than those present in the dispersion medium. The at least one other compound is chosen such that the graft polymer remains in the dispersed state in the liquid fatty phase.

The organic liquid dispersion medium is present in the liquid fatty phase of the at least one first composition according to the present disclosure because of the introduction of the graft polymer dispersion obtained into the at least one first composition.

The liquid fatty phase may, for example, predominantly comprise at least one organic liquid compound (or oil) as defined below. For instance, the liquid fatty phase can be an organic liquid phase that is nonaqueous and immiscible with water at room temperature (25° C.).

As used herein, the term “organic liquid compound” is understood to mean a nonaqueous compound which is in the liquid state at room temperature (25° C.) and which therefore flows under its own weight.

As used herein, the expression “silicone compound” is understood to mean a compound comprising at least one silicon atom.

Among the liquid organic compounds or oils which may be present in the organic liquid dispersion medium, non-limiting mention may be made of:
the organic liquid, for instance nonsilicone- or silicone-based, compounds having an overall solubility parameter according to the Hansen solubility space of less than or equal to 18 (MPa)^{1/2}, such as less than or equal to 17 (MPa)^{1/2},
the monoalcohols having an overall solubility parameter according to the Hansen solubility space of less than or equal to 20 (MPa)^{1/2}; and
mixtures thereof.

The overall solubility parameter δ according to the Hansen solubility space is defined in the article “Solubility Parameter Values” by Eric A. Grulke in the book “Polymer Handbook,” 3rd edition, chapter VII, p. 519-559 by the equation:
δ=(d_{D}²+d_{P}²+d_{H}²)^{1/2}
in which
d_{D} is the LONDON dispersion forces resulting from the formation of dipoles induced during molecular impacts,
d_{P} is the DEBYE interactive forces between permanent dipoles, and
d_{H} is the specific interactive forces (hydrogen bond, acid/base or donor/acceptor type, and the like). The definition of the solvents in the solubility space according to Hansen is described in the article by C. M. Hansen “The Three Dimensional Solubility Parameters” J. Paint Technol. 39, 105 (1967).

Among the organic liquid, for instance non silicone- or silicone-based, compounds having an overall solubility parameter according to the Hansen solubility space of less than or equal to 18 (MPa)^{1/2} that may be used, non-limiting mention may be made of liquid fatty substances, such as oils, which may be chosen from natural or synthetic, carbonaceous, hydrocarbonaceous, fluorinated or silicone, optionally branched, oils, alone or as a mixture.

As used herein, the term “oil” is understood to mean any nonaqueous medium that is liquid at room temperature (25° C.) and atmospheric pressure (760 mmHg), that is compatible with application to the skin, the mucous membranes (lips) and/or the superficial body growths (nails, eyelashes, eyebrows, hair).

Among the oils that may be used as disclosed herein, non-limiting mention may be made of the vegetable oils formed by fatty acid esters of polyols, for instance triglycerides, such as sunflower, sesame or rapeseed oil, or esters derived from long chain acids or alcohols (that is to say having from 6 to 20 carbon atoms), for instance the esters of formula RCOOR′ wherein R is chosen from the remainders of higher fatty acids comprising from 7 to 19 carbon atoms and R′ is chosen from hydrocarbon chains comprising from 3 to 20 carbon atoms, such as palmitates, adipates and benzoates, for example diisopropyl adipate.

Further non-limiting mention may be made of linear, branched and/or cyclic, optionally volatile, alkanes, for instance paraffin oils, petroleum jelly or hydrogenated polyisobutylene, isododecane or “ISOPARS”, volatile isoparaffins. Non-limiting mention may also be made of esters, ethers and ketones.

Still further non-limiting mention may be made of silicone oils such as polydimethylsiloxanes and polymethylphenylsiloxanes, optionally substituted with aliphatic and/or aromatic, optionally fluorinated, groups, or with functional groups such as hydroxyl, thiol and/or amine groups, and volatile, such as cyclic, silicone oils.

For example, the at least one first composition may comprise a volatile oil.

As used herein, the term “volatile oil” is understood to mean any nonaqueous medium capable of evaporating on the skin or the lips, in less than one hour, having, for example, a vapor pressure, at room temperature and atmospheric pressure, ranging from 10⁻³ to 300 mmHg (0.13 Pa to 40 000 Pa).

For further example, the at least one first composition according to the present disclosure may comprise a volatile oil in an amount ranging from 1% to 70% by weight, relative to the total weight of the at least one first composition, such as ranging from 5% to 50% by weight, for instance ranging from 10% to 35% by weight.

Among the volatile silicone oils that may be used as disclosed herein, non-limiting mention may be made of linear or cyclic silicones comprising from 2 to 7 silicon atoms, these silicones optionally comprising alkyl or alkoxy groups comprising from 1 to 10 carbon atoms. Non-limiting examples further include: octamethylcyclotetrasiloxane, decamethylcyclopentasiloxane, dodecamethylcyclohexasiloxane, heptamethylhexyltrisiloxane, heptamethyloctyltrisiloxane, octamethyltrisiloxane, decamethyltetrasiloxane and mixtures thereof.

Further among the volatile nonsilicone oils, non-limiting mention may be made of isododecane or “ISOPARS”, volatile isoparaffins, isohexadecane and isodecane.

The at least one first composition can comprise a non-volatile oil, for instance in an amount ranging from 1% to 80% by weight, relative to the total weight of the at least one first composition, for example ranging from 5% to 60% by weight, and such as ranging from 10% to 50% by weight.

Among the non-volatile silicone oils that may be used, non-limiting mention may be made of non-volatile polydialkylsiloxanes such as non-volatile polydimethylsiloxanes (PDMS); polydimethylsiloxanes comprising alkyl, alkoxy or phenyl groups that are pendant or at the silicone chain end, groups having from 2 to 24 carbon atoms; phenylated silicones such as phenyl trimethicones, phenyl dimethicones, phenyl trimethylsiloxy diphenylsiloxanes, diphenyl dimethicones, diphenyl methyldiphenyl trisiloxanes, polymethylphenylsiloxanes; polysiloxanes modified with fatty acids (such as C₈-C₂₀ fatty acids), fatty alcohols (such as C₈-C₂₀ fatty alcohols) or polyoxyalkylenes (such as polyoxyethylene and/or polyoxypropylene); aminated polysiloxanes; polysiloxanes with hydroxyl groups; fluorinated polysiloxanes comprising a fluorinated group that is pendant or at the silicone chain end comprising from 1 to 12 carbon atoms of which some or all of the hydrogens are substituted with fluorine atoms; and mixtures thereof.

By way of non-limiting example, there may be mentioned, among the nonsilicone organic liquid compounds having an overall solubility parameter according to the Hansen solubility space of less than or equal to 18 (MPa)^{1/2}:
linear, branched or cyclic esters comprising at least 6 carbon atoms, for instance ranging from 6 to 30 carbon atoms;
ethers having at least 6 carbon atoms, for instance from 6 to 30 carbon atoms; and
ketones having at least 6 carbon atoms, for instance from 6 to 30 carbon atoms.

As used herein, the expression “liquid monoalcohols having an overall solubility parameter according to the Hansen solubility space of less than or equal to 20 (MPa)^{1/2}” is understood to mean aliphatic liquid fatty monoalcohols comprising from 6 to 30 carbon atoms, the hydrocarbon chain comprising no substituent group. As monoalcohols that may be used according to the present disclosure, non-limiting examples include oleyl alcohol, decanol and linoleyl alcohol.

According to one embodiment of the present disclosure, the liquid fatty phase may be a nonsilicone liquid fatty phase.

As used herein, the expression “nonsilicone liquid fatty phase” is understood to mean a fatty phase comprising at least one nonsilicone liquid organic compound or oil such as those mentioned above, wherein the at least one nonsilicone compound is predominantly present in the liquid fatty phase, that is to say at least 50% by weight, such as from 50% to 100% by weight, for instance from 60% to 100% by weight (for example from 60% to 99% by weight), or alternatively from 65% to 100% by weight (for example from 65% to 95% by weight), relative to the total weight of the liquid fatty phase.

The nonsilicone liquid organic compounds may be chosen from, by way of non-limiting example:
nonsilicone liquid organic compounds having an overall solubility parameter according to the Hansen solubility space of less than or equal to 18 (MPa)^{1/2},
monoalcohols having an overall solubility parameter according to the Hansen solubility space of less than or equal to 20 (MPa)^{1/2}; and
mixtures thereof.

The nonsilicone liquid fatty phase may optionally comprise silicone liquid organic compounds or oils such as those mentioned above, which can be present in an amount of less than or equal to 50% by weight, for instance ranging from 0.1% to 40% by weight, or even ranging from 1% to 35% by weight, or alternatively ranging from 5% to 30% by weight, relative to the total weight of the liquid fatty phase.

According to one embodiment of the present disclosure, the nonsilicone liquid fatty phase does not contain silicone organic liquid compounds or oils.

When the liquid fatty phase is a nonsilicone liquid fatty phase, the macromonomers present in the graft polymer can be, for example, carbonaceous macromonomers as described below.

For instance, when the liquid fatty phase is a nonsilicone liquid fatty phase, the graft polymer present in the at least one first composition can be a nonsilicone graft polymer.

As used herein, the expression “nonsilicone graft polymer” is understood to mean a graft polymer predominantly comprising a carbonaceous macromonomer and optionally comprising at most 7% by weight of the total weight of the polymer, such as at most 5% by weight, or is even free of silicone macromonomer.

According to another embodiment of the present disclosure, the liquid fatty phase may be a silicone liquid fatty phase.

As used herein, the expression “silicone liquid fatty phase” is understood to mean a fatty phase comprising at least one silicone organic liquid compound or silicone oil such as those described above, wherein the silicone compounds are predominantly present in the liquid fatty phase, that is to say present in an amount of at least 50% by weight, for instance from 50% to 100% by weight, such as from 60% to 100% by weight (for example from 60% to 99% by weight), or alternatively from 65% to 100% by weight (for example from 65% to 95% by weight), relative to the total weight of the liquid fatty phase.

The silicone organic liquid compounds may be chosen, by way of non-limiting example, from:
organic liquid, such as nonsilicone or silicone, compounds having an overall solubility parameter according to the Hansen solubility space of less than or equal to 18 (MPa)^{1/2}, for instance less than or equal to 17 (MPa)^{1/2}.

The silicone liquid fatty phase may optionally comprise nonsilicone organic liquid compounds or oils, as described above, which may be present in an amount of less than or equal to 50% by weight, for instance ranging from 0.1 to 40% by weight, or even ranging from 1 to 35% by weight, or alternatively ranging from 5 to 30% by weight, relative to the total weight of the liquid fatty phase.

According to one embodiment of the present disclosure, the silicone liquid fatty phase does not contain nonsilicone organic liquid compounds.

When the liquid fatty phase is a silicone liquid fatty phase, the macromonomers present in the graft polymer can be, for example, silicone macromonomers as described below. For instance, when the liquid fatty phase is a silicone liquid fatty phase, the graft polymer present in the at least one first composition can be for example, a silicone graft polymer.

As used herein, the expression “silicone graft polymer” is understood to mean a graft polymer predominantly comprising a silicone macromonomer and optionally comprising at most 7% by weight of the total weight of the polymer, for instance at most 5% by weight, or is even free of, carbonaceous macromonomer.

The choice of the monomers constituting the backbone of the polymer, of the macromonomers, the molecular weight of the polymer, the proportion of the monomers and of the macromonomers can be made according to the organic liquid dispersion medium so as to obtain, for example, a dispersion of particles of graft polymers, such as a stable dispersion, it being possible for this choice to be made by persons skilled in the art.

As used herein, the expression “stable dispersion” is understood to mean a dispersion which is not capable of forming a solid deposit or of solid/liquid phase separation, for instance after centrifugation, for example, at 4000 revolutions/minute for 15 minutes.

The graft ethylene polymer forming the particles in dispersion therefore comprises a backbone that is insoluble in the dispersion medium and a part that is soluble in the dispersion medium.

The graft ethylene polymer can be a random polymer.

As used herein, the expression “graft ethylene polymer” is understood to mean a polymer capable of being obtained by free-radical polymerization:
of at least one ethylene monomer,
with at least one macromonomer, in an organic polymerization medium.

As used herein, the expression “graft acrylic polymer” is understood to mean a polymer capable of being obtained by free-radical polymerization:
of at least one acrylic monomer, and optionally of at least one additional nonacrylic vinyl monomers,
with at least one macromonomer, in an organic polymerization medium.

For example, the acrylic monomers can be present in an amount ranging from 50% to 100% by weight, for instance, from 55% to 100% by weight (such as from 55% to 95% by weight), for instance from 60% to 100% by weight (such as from 60% to 90% by weight) of the acrylic monomer and optional nonacrylic vinyl monomer mixture.

By way of non-limiting example, the acrylic monomers can be chosen from monomers whose homopolymer is insoluble in the dispersion medium considered, that is to say that the homopolymer is in solid (or undissolved) form at a concentration of greater than or equal to 5% by weight at room temperature (20° C.) in the dispersion medium.

As used herein, the expression “macromonomer having a polymerizable end group” is understood to mean any polymer comprising at only one of its ends a polymerizable end group capable of reacting during the polymerization reaction with the acrylic monomers and optionally with the additional nonacrylic vinyl monomers constituting the backbone. The macromonomer makes it possible to form the side chains of the graft acrylic polymer. The polymerizable group of the macromonomer can be, for example, a group with ethylenic unsaturation capable of polymerizing by the free-radical route with the monomers constituting the backbone.

As used herein, the expression “carbonaceous macromonomer” is understood to mean a nonsilicone macromonomer, for example, an oligomeric macromonomer obtained by polymerization of nonsilicone monomer(s) with ethylenic unsaturation, and mainly by polymerization of acrylic and/or nonacrylic vinyl monomers.

As used herein, the expression “silicone macromonomer” is understood to mean an organopolysiloxane macromonomer, for instance a polydimethylsiloxane macromonomer.

For example, the macromonomer can chosen from macromonomers whose homopolymer is soluble in the dispersion medium considered, that is to say completely dissolved at a concentration of greater than or equal to 5% by weight and at room temperature in the dispersion medium.

Thus, the graft acrylic polymer comprises a backbone (or main chain) comprising a succession of acrylic units resulting from the polymerization, for instance, of at least one acrylic monomer and side chains (or grafts) derived from the reaction of the macromonomers, the side chains being covalently linked to the main chain.

The backbone (or main chain) is insoluble in the dispersion medium considered while the side chains (or grafts) are soluble in the dispersion medium.

As used herein, the expression “acrylic monomer” is understood to mean, monomers chosen from (meth)acrylic acid, (meth)acrylic acid esters (also called (meth)acrylates), (meth)acrylic acid amides (also called (meth)acrylamides).

Among the acrylic monomers that are capable of being used to form the insoluble backbone of the polymer, non-limiting mention may be made of, alone or as a mixture, the following monomers and their salts:
(i) the (meth)acrylates of formula: 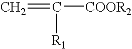
   wherein:
R₁ is chosen from a hydrogen atom and a methyl group;
R₂ is a group chosen from:
   linear and branched alkyl groups comprising from 1 to 6 carbon atoms, wherein the groups may comprise in their chains at least one heteroatom chosen from O, N and S atoms; and/or may comprise at least one substituent chosen from —OH, halogen atoms (such as F, Cl, Br, I) and —NR′R″ wherein R′ and R″, which may be identical or different, are chosen from linear and branched C₁-C₄ alkyls; and/or may be substituted with at least one polyoxyalkylene group, such as with C₂-C₄ alkylene, for instance polyoxyethylene and/or polyoxypropylene, the polyoxyalkylene group comprising the repetition of 5 to 30 oxyalkylene units;
   cyclic alkyl groups comprising from 3 to 6 carbon atoms, wherein the groups may comprise in their chains at least one heteroatom chosen from O, N and S atoms, and/or may comprise at least one substituent chosen from OH groups and halogen atoms (such as F, Cl, Br, I).

By way of non-limiting examples of R₂, there may be mentioned methyl, ethyl, propyl, butyl, isobutyl, methoxyethyl, ethoxyethyl, methoxypolyoxyethylene 350 ethyleneoxide, trifluoroethyl, 2-hydroxyethyl, 2-hydroxypropyl, dimethylaminoethyl, diethylaminoethyl, and dimethylaminopropyl groups.
(ii) the (meth)acrylamides of formula: 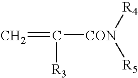
   wherein:
R₃ is chosen from a hydrogen atom and a methyl group;
R₄ and R₅, which may be identical or different are chosen from hydrogen atoms and linear and branched alkyl groups comprising from 1 to 6 carbon atoms, which may comprise at least one substituent chosen from —OH, halogen atoms (such as F, Cl, Br, I) and —NR′R″ wherein R′ and R″, which may be identical or different, are chosen from linear and branched C₁-C₄ alkyls; or
R₄ is a hydrogen atom and R₅ is a 1,1-dimethyl-3-oxobutyl group.

By way of non-limiting examples of alkyl groups which may constitute R₄ and R₅, there may be mentioned n-butyl, t-butyl, n-propyl, dimethylaminoethyl, diethylaminoethyl and dimethylaminopropyl groups.
(iii) the (meth)acrylic monomers comprising at least one functional groups chosen from carboxylic, phosphoric and sulphonic acid functional groups, such as acrylic acid, methacrylic acid, and acrylamidopropanesulphonic acid.

Among these acrylic monomers, non-limiting mention may be made of, for example, methyl, ethyl, propyl, butyl and isobutyl (meth)acrylates; methoxyethyl or ethoxyethyl (meth)acrylates; trifluoromethyl methacrylate; dimethylaminoethyl methacrylate, diethylaminoethyl methacrylate, 2-hydroxypropyl methacrylate, 2-hydroxyethyl methacrylate, 2-hydroxypropyl acrylate, 2-hydroxyethyl acrylate; dimethylaminopropylmethacrylamide; (meth)acrylic acid; and their salts; and mixtures thereof.

For example, the acrylic monomers can be chosen from methyl acrylate, methoxyethyl acrylate, methyl methacrylate, 2-hydroxyethyl methacrylate, (meth)acrylic acid, dimethylaminoethyl methacrylate, and mixtures thereof.

Among the additional nonacrylic vinyl monomers that can be used as disclosed herein, there may be mentioned, by way of non-limiting example:
the vinyl esters of formula: R₆—COO—CH═CH₂
   wherein R₆ is chosen from linear and branched alkyl groups comprising from 1 to 6 atoms, or a cyclic alkyl group comprising from 3 to 6 carbon atoms and/or an aromatic group, for example of the benzene, anthracene and naphthalene type;
the nonacrylic vinyl monomers comprising at least one functional group chosen from carboxylic, phosphoric and sulphonic acid functional groups, such as crotonic acid, maleic anhydride, itaconic acid, fumaric acid, maleic acid, styrenesulphonic acid, vinylbenzoic acid, vinylphosphoric acid, and their salts;
the nonacrylic vinyl monomers comprising at least one tertiary amine functional group, such as 2-vinylpyridine and 4-vinylpyridine;
and mixtures thereof.

According to one embodiment of the present disclosure, the graft polymer comprises (meth)acrylic acid.

For example, the acrylic monomers present in the graft polymer can comprise at least (meth)acrylic acid, such as at least (meth)acrylic acid and at least one monomer chosen from the (meth)acrylates and (meth)acrylamides described above in points (i) and (ii). For instance, the acrylic monomers comprise at least (meth)acrylic acid and at least one monomer chosen from C₁-C₃ alkyl (meth)acrylates. The (meth)acrylic acid may be present in an amount of at least 5% by weight, relative to the total weight of the polymer (such as ranging from 5% to 80% by weight), for example at least 10% by weight (such as ranging from 10% by weight to 70% by weight), for example, at least 15% by weight (such as ranging from 15% to 60% by weight).

Among the salts that may be used as disclosed herein, non-limiting mention may be made of those obtained by neutralizing the acid groups with the aid of inorganic bases such as sodium hydroxide, potassium hydroxide, ammonium hydroxide, or organic bases of the alkanolamine type such as monoethanolamine, diethanolamine, triethanolamine or 2-methyl-2-amino-1-propanol. Non-limiting mention may also be made of the salts formed by neutralizing the tertiary amine units, for example with the aid of an inorganic or organic acid. Among the inorganic acids, non-limiting mention may be made of sulphuric acid, hydrochloric acid, hydrobromic acid, hydriodic acid, phosphoric acid and boric acid. Among the organic acids, non-limiting mention may be made of the acids comprising at least one group chosen from carboxylic, sulphonic and phosphonic groups. These may be linear, branched or cyclic aliphatic acids or alternatively aromatic acids. These acids may additionally comprise at least one heteroatom chosen from O and N atoms, for example in the form of hydroxyl groups. There may also be mentioned, in a non-limiting manner, for example, acetic acid or propionic acid, terephthalic acid, and citric acid and tartaric acid.

According to one embodiment of the present disclosure, the graft ethylene polymer does not comprise additional nonacrylic vinyl monomers as described above. In this embodiment, the insoluble backbone of the graft ethylene polymer is formed solely of acrylic monomers as described above.

It is understood that these nonpolymerized acrylic monomers may be soluble in the dispersion medium considered, but the polymer formed with these monomers is insoluble in the dispersion medium.

According to another embodiment of the present disclosure, the graft ethylene polymer is capable of being obtained by free-radical polymerization in an organic polymerization medium:
of a principal acrylic monomer chosen from C₁-C₃ alkyl (meth)acrylates, alone or as a mixture, and optionally of at least one additional acrylic monomer chosen from acrylic acid, methacrylic acid and the alkyl (meth)acrylates of formula (I) defined below, and their salts, in order to form the insoluble backbone; and
of at least one silicone macromonomer comprising a polymerizable end group as defined above.

As principal acrylic monomer, there may be used methyl acrylate, methyl methacrylate, ethyl acrylate, ethyl methacrylate, n-propyl acrylate, n-propyl methacrylate, isopropyl acrylate and isopropyl methacrylate, and mixtures thereof.

For example, in one embodiment of the present disclosure, the acrylic monomer is chosen from methyl acrylate, methyl methacrylate and ethyl methacrylate.

The additional acrylic monomers may be chosen from:
(meth)acrylic acid and its salts,
the (meth)acrylates of formula (I) and their salts; 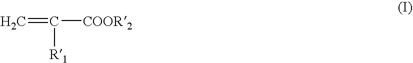
   wherein:
R′₁ is chosen from a hydrogen atom and a methyl group;
R′₂ is chosen from
linear and branched alkyl groups comprising from 1 to 6 carbon atoms, the groups comprising in their chains at least one oxygen atom and/or comprising at least one substituent chosen from —OH groups, halogen atoms (such as F, Cl, Br, I) and —NR′R″ wherein R′ and R″, which may be identical or different, are chosen from linear and branched C₁-C₃ alkyls;
a cyclic alkyl group comprising from 3 to 6 carbon atoms, wherein the group may comprise in its chain at least one oxygen atom and/or may comprise at least one substituent chosen from OH and halogen atoms (such ah F, Cl, Br, I);
and mixtures thereof.

By way of non-limiting examples of R′₂, there may be mentioned the methoxyethyl, ethoxyethyl, trifluoroethyl; 2-hydroxyethyl, 2-hydroxypropyl, dimethylaminoethyl, diethylaminoethyl, and dimethylaminopropyl groups.

Further among these additional acrylic monomers, there may be mentioned, for example, (meth)acrylic acid, methoxyethyl or ethoxyethyl (meth)acrylates; trifluoroethyl methacrylate; dimethylaminoethyl methacrylate, diethylaminoethyl methacrylate, 2-hydroxypropyl methacrylate, 2-hydroxyethyl methacrylate, 2-hydroxypropyl acrylate, 2-hydroxyethyl acrylate, their salts, and mixtures thereof.

In one embodiment of the present disclosure, acrylic acid and methylacrylic acid are used.

The macromonomers comprise at one of the chain ends a polymerizable end group capable of reacting, during the polymerization, with the acrylic monomers and optionally the additional vinyl monomers to form the side chains of the graft ethylene polymer. The said polymerizable end group may be, for example, a vinyl or (meth)acrylate (or (meth)acryloyl) group, such as a (meth)acrylate group.

The macromonomers can be chosen from, for instance, the macromonomers whose homopolymer has a glass transition temperature (Tg) of less than or equal to 25° C., such as ranging from −100° C. to 25° C., for example ranging from −80° C. to 0° C.

The macromonomers have a weight-average molecular mass of greater than or equal to 200, for instance greater than or equal to 300, such as greater than or equal to 500, and for example greater than 600. For example, the macromonomers have a weight-average molecular mass (Mw) ranging from 200 to 100,000, such as ranging from 500 to 50 000, for instance ranging from 800 to 20,000, and from 800 to 10,000, such as ranging from 800 to 6,000.

As disclosed herein, the weight-average (Mw) and number-average (Mn) molar masses are determined by gel permeation liquid chromatography (solvent THF, calibration curve established with linear polystyrene standards, refractometric detector).

Among the carbonaceous macromonomers that may be used herein, there may be mentioned, by way of non-limiting example:
(i) the linear or branched C₈-C₂₂ alkyl (meth)acrylate homopolymers and copolymers having a polymerizable end group chosen from vinyl or (meth)acrylate groups among which non-limiting mention may be made of, for instance: macromonomers of poly(2-ethylhexyl acrylate) with a mono(meth)acrylate end; macromonomers of poly(dodecyl acrylate) or poly(dodecyl methacrylate) with a mono(meth)acrylate end; macromonomers of poly(stearyl acrylate) or of poly(stearyl methacrylate) with a mono(meth)acrylate end.

Such macromonomers are described for example, in European Patent Nos. EP 895467 and EP 96459 and in the article Gillman K. F., Polymer Letters, Vol. 5, page 477-481 (1967). Further non-limiting mention may be made of the macromonomers based on poly(2-ethylhexyl acrylate) or poly(dodecyl acrylate) with a mono(meth)acrylate end.
(ii) the polyolefins having an end group with ethylenic unsaturation, for instance having a (meth)acrylate end group. As non-limiting examples of such polyolefins, the following macromonomers can be mentioned, for instance, it being understood that they have a (meth)acrylate end group: polyethylene macromonomers, polypropylene macromonomers, polyethylene/polypropylene copolymer macromonomers, polyethylene/polybutylene copolymer macromonomers, polyisobutylene macromonomers; polybutadiene macromonomers; polyisoprene macromonomers; polybutadiene macromonomers, poly(ethylene/butylene)-isoprene macromonomers. Such macromonomers are described for example in U.S. Pat. No. 5,625,005, which mentions ethylene/butylene and ethylene/propylene macromonomers with a (meth)acrylate reactive end group. In addition, non-limiting mention may be made of poly(ethylene/butylene) methacrylate, such as that marketed under the name Kraton Liquid L-1253 by Kraton Polymers.

Among the silicone macromonomers that may be used herein, non-limiting mention may be made of, for instance, polydimethylsiloxanes with a mono(meth)acrylate end group, such as those of formula (II): 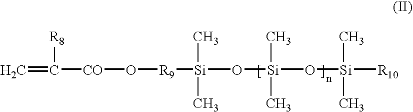
wherein R₈ is chosen from a hydrogen atom and a methyl group; R₉ is chosen from divalent hydrocarbon groups having from 1 to 10 carbon atoms and optionally comprises one or two ether bonds —O—; R₁₀ is chosen from alkyl groups comprising from 1 to 10 carbon atoms, such as from 2 to 8 carbon atoms; n is an integer ranging from 1 to 300, for instance ranging from 3 to 200, such as ranging from 5 to 100.

As non-limiting examples of silicone macromonomers, there may be mentioned monomethacryloxypropyl polydimethylsiloxanes such as those marketed under the name PS560-K6 by the company United Chemical Technologies Inc. (UCT) or under the name MCR-M17 by the company Gelest Inc.

For instance, the polymerized macromonomer (constituting the side chains of the graft polymer) can be present in an amount ranging from 0.1% to 15% by weight, relative to the total weight of the polymer, such as from 0.2% to 10% by weight, and from 0.3% to 8% by weight.

Further among the graft ethylene polymer dispersed in a nonsilicone liquid fatty phase, there may be used, in a non-limiting manner, those obtained by polymerization:
of methyl acrylate and the polyethylene/polybutylene macromonomer with a methacrylate end group (for instance Kraton L-1253), such as in a solvent chosen from isododecane, iononyl isononanoate, octyldodecanol, diisostearyl malate, a C₁₂-C₁₅ alkyl benzoate (such as Finsolv TN);
of methoxyethyl acrylate and the polyethylene/polybutylene macromonomer with a methacrylate end group (such as Kraton L-1253), for instance in isododecane;
of the methyl acrylate/methyl methacrylate monomers and the polyethylene/polybutylene macromonomer with a methacrylate end group (such as Kraton L-1253), for instance in isododecane;
of the methyl acrylate/acrylic acid macromonomers and the polyethylene/polybutylene macromonomer with a methacrylate end group (such as Kraton L-1253), for instance in isododecane;
of the methyl acrylate/dimethylaminoethyl methacrylate monomers and the polyethylene/polybutylene macromonomer with a methacrylate end group (for instance Kraton L-1253), such as isododecane;
of the methyl acrylate/2-hydroxyethyl methacrylate monomers and the polyethylene/polybutylene macromonomer with a methacrylate end group (such as Kraton L-1253), for instance in isododecane.

Among the graft acrylic polymers dispersed in a silicone liquid fatty phase, there may be used, by way of non-limiting example, those obtained by polymerization:
of methyl acrylate and the monomethacryloxypropylpolydimethylsiloxane macromonomer having a weight-average molecular weight ranging from 800 to 6,000, for instance in decamethylcyclopentasiloxane or phenyltrimethicone;
of methyl acrylate, acrylic acid and the monomethacryloxypropylpolydimethylsiloxane macromonomer having a weight-average molecular weight ranging from 800 to 6,000, for instance in decamethylcyclopentasiloxane or phenyltrimethicone.

For example, the graft polymer can have a weight-average molecular mass (Mw) ranging from 10,000 and 300,000, such as from 20,000 to 200,000, for instance from 25,000 to 150,000.

By virtue of the abovementioned characteristics, in a given organic dispersion medium, the polymers have the capacity to replicate on themselves, thus forming particles of substantially spherical shape, with, on the periphery of these particles, the side chains displayed, which ensure the stability of these particles. Such particles resulting from the characteristics of the graft polymer can have the characteristic feature of not agglomerating in the medium and therefore of becoming self-stabilized and of forming, for example, a stable dispersion of polymer particles.

In one embodiment, the graft ethylene polymers of the dispersion may form nanometric particles having a mean size ranging from 10 nm to 400 nm, such as from 20 nm to 200 nm.

Because of this very small size, the graft polymer particles in dispersion are particularly stable and therefore not very likely to form agglomerates. The graft polymer dispersion can therefore be a stable dispersion and may not form sediments when it is placed for a prolonged period (for example 24 hours) at room temperature (25° C.).

For instance, the dispersion of graft polymer particles can be present in a polymer dry matter (or dry extract) amount ranging from 40% to 70% by weight of dry matter, such as ranging from 45% to 65% by weight.

The graft polymer particle dispersion may be prepared by a method comprising a step of free-radical copolymerization, in an organic polymerization medium, of at least one acrylic monomer as defined above, with at least one macromonomer as defined above.

As indicated above, the organic liquid dispersion medium may be the same or different from the polymerization medium.

Conventionally, the copolymerization can be carried out in the presence of at least one polymerization initiator. The polymerization initiators can be free-radical initiators. In general, such a polymerization initiator can be chosen from peroxidized organic compounds such as dilauroyl peroxide, dibenzoyl peroxide, tert-butyl peroxy-2-ethylhexanoate; diazotized compounds such as azobisisobutyronitrile and azobisdimethylvaleronitrile.

The reaction can also be initiated with the aid of photoinitiators or with radiation such as UV radiation, neutrons or with plasma.

In general, to carry out this method, at least a portion of the organic polymerization medium, a portion of the acrylic and/or additional vinyl monomers, which will constitute, after polymerization, the insoluble backbone, the entire macromonomer (which will constitute the side chains of the polymer) and a portion of the polymerization initiator are introduced into a reactor having a size appropriate for the quantity of polymer which will be produced. At this introduction stage, the reaction medium forms a relatively homogeneous medium.

The reaction medium is then stirred and heated to a temperature in order to obtain polymerization of the monomers and macromonomers. After a certain time, the initially homogeneous and clear medium leads to a dispersion having a milky appearance. A mixture comprising the remaining portion of monomers and the polymerization initiator is then added. After a suitable time during which the mixture is heated, with stirring, the medium becomes stabilized in the form of a milky dispersion, the dispersion comprising particles of stabilized polymers in the medium in which they were created, the stabilization being due to the presence, in the polymer, of side chains that are soluble in the dispersion medium.

The at least one first composition according to the present disclosure makes it possible to obtain a deposit, such as a make-up deposit, on keratin materials, for instance on the skin or the lips having good gloss and good transfer-resistant properties.

The at least one first composition according to the present disclosure also makes it possible to obtain a deposit on keratin materials, for example on the skin or the lips not exhibiting a sensation of dryness and tightness: the deposit thus obtained is therefore comfortable over time for the user.

The graft polymer described above may be present in the at least one first composition according to the present disclosure in an amount ranging from 0.1% to 70% by weight, relative to the total weight of the at least one first composition, for instance ranging from 0.5% to 50% by weight, such as ranging from 1% to 40% by weight.

The present examples illustrate the preparation of polymers in accordance with the present disclosure, capable of forming a dispersion of particles in an organic medium considered.

In these examples, the weight-average (Mw) and number-average (Mn) molar masses of the polymer, the glass transition temperature of the polymer, the amount of dry matter (or dry extract) present in the dispersion and the size of the particles of polymers are determined after preparation of the dispersion. The weight-average (Mw) and number-average (Mn) molar masses are determined by gel permeation liquid chromatography (solvent THF, calibration curve established with linear polystyrene standards, refractometric detector).

The measurement of the glass transition temperature (Tg) is carried out according to the ASTM D3418-97 standard, by differential enthalpic analysis (DSC, Differential Scanning Calorimetry) on a calorimeter, over a temperature range ranging from −100° C. and +150° C. at a heating rate of 10° C./min in 150 μl aluminium crucibles. The preparation of the crucibles is carried out in the following manner: 100 μl of the dispersion obtained are introduced into a 150 μl aluminium crucible and the solvent is left to evaporate for 24 hours at room temperature and at a relative humidity of 50%. The operation is repeated and then the crucible is introduced into the Mettler DSC30 calorimeter.

The amount of dry matter (or dry extract), i.e., the amount of non-volatile material present, can be measured in various ways: there may be mentioned, by way of non-limiting example, the methods based on drying in an oven or the methods based on drying by exposure to infrared radiation. For instance, the amount of dry matter present can be measured by heating the sample with infrared rays having a wavelength of 2 μm to 3.5 μm. The substances contained in the composition that possess a high vapor pressure evaporate under the effect of this radiation.

The measurement of the loss of weight of the sample makes it possible to determine the amount of dry extract present in the composition. These measurements are carried out by means of a commercial LP16 infrared dessicator from Mettler. This technique is fully described in the documentation for the apparatus supplied by Mettler.

The measurement protocol is as follows: about 1 g of the composition is spread in a metal dish. The latter, after introduction into the dessicator, is subjected to a reference temperature of 120° C. for one hour. The wet sample mass, corresponding to the initial mass, and the dry sample mass, corresponding to the mass after exposure to radiation, are measured by means of a precision balance. The amount of dry matter present is calculated in the following manner:

$$Dry extract = 100 \times ( dry mass / wet mass ) .$$

The particle sizes can be measured by various techniques: there may be mentioned, for instance, (dynamic and static) light scattering techniques, methods based on the Coulter counter, measurements based on sedimentation speed (linked to the size via Stokes law) and microscopy. These techniques can make it possible to measure a particle diameter and, for some of them, a particle size distribution. For example, the sizes and size distributions of the particles of the compositions according to the present disclosure can be measured by static light scattering by means of a commercial particle size analyser of the MasterSizer 2000 type from Malvern. The data are processed on the basis of the Mie light-scattering theory. This theory, which is accurate for isotropic particles, makes it possible to determine, in the case of nonspherical particles, an “effective” particle diameter. This theory is described, for instance, in the book by Van de Hulst, H. C., “Light Scattering by Small Particles”, chapters 9 and 10, New York, 1957.

The composition is characterized by its mean “effective” diameter by volume D[4.3], defined in the following manner:

$$D [ 4.3 ] = \frac{{\sum }_{i} {V}_{i} \cdot {d}_{i}}{{\sum }_{i} {V}_{i}}$$

where Vᵢ is the volume of particles with an effective diameter dᵢ. This parameter is described in the article in the technical documentation for the particle size analyser. The measurements are carried out at 25° C. on a dilute particle dispersion obtained from the composition in the following manner: 1) dilution by a factor of 100 with water, 2) homogenization of the solution, 3) solution allowed to stand for 18 hours, 4) recovery of the whitish homogeneous supernatant. The “effective” diameter is obtained by taking a refractive index of 1.33 for water and a mean refractive index of 1.42 for the particles.

The at least one first composition according to the present disclosure may also comprise at least one fatty substance that is solid at room temperature, chosen for instance from waxes, pasty fatty substances, gums and mixtures thereof. These fatty substances may be of animal, plant, mineral or synthetic origin.

As used herein, the expression “pasty fatty substance” is understood to mean a lipophilic compound comprising, at the temperature of 23° C., a liquid fraction and a solid fraction. The expression “pasty fatty substance” is also understood to mean polyvinyl laurate.

The pasty compound can have, for example, a hardness at 20° C. ranging from 0.001 MPa to 0.5 MPa, such as from 0.002 MPa to 0.4 MPa.

The hardness is measured according to a method based on penetration of a probe into a sample of compound and for example, with the aid of a texture analyser (for example TA-XT2i from Rheo) equipped with a stainless steel cylinder 2 mm in diameter. The measurement of hardness is carried out at 20° C. at the center of 5 samples. The cylinder is introduced into each sample at a pre-speed of 1 mm/s and then at a measurement speed of 0.1 mm/s, the depth of penetration being 0.3 mm. The measured value of the hardness is that of the maximum peak.

The liquid fraction of the pasty compound measured at 23° C. can be, for example, present in an amount ranging from 9% to 97% by weight of the compound. The fraction that is liquid at 23° C. can be present in an amount ranging from 15% to 85%, such as from 40% to 85% by weight. The liquid fraction by weight of the pasty compound at 23° C. is equal to the ratio of the enthalpy of fusion consumed at 23° C. to the enthalpy of fusion of the pasty compound.

The enthalpy of fusion of the pasty compound is the enthalpy consumed by the compound to pass from the solid state to the liquid state. The pasty compound is said to be in the solid state when the whole of its mass is in solid crystalline form. The pasty compound is said to be in the liquid state when the whole of its mass is in liquid form. The enthalpy of fusion of the pasty compound is equal to the surface under the curve of the thermogram obtained with the aid of a differential scanning calorimeter (D.S.C.), such as the calorimeter sold under the name MDSC 2920 by the company TA instrument, with a temperature rise of 5° C. or 10° C. per minute according to the ISO 11357-3:1999 standard. The enthalpy of fusion of the pasty compound is the quantity of energy necessary to cause the compound to pass from the solid state to the liquid state. It is expressed in J/g. The enthalpy of fusion consumed at 23° C. is the quantity of energy absorbed by the sample in order to pass from the solid state to the state in which it exists at 23° C. comprising a liquid fraction and a solid fraction.

The liquid fraction of the pasty compound measured at 32° C. can be present in an amount, for example, ranging from 30% to 100% by weight of the compound, such as from 80% to 100%, for instance from 90% to 100% by weight of the compound. When the liquid fraction of the pasty compound measured at 32° C. is equal to 100%, the temperature at the end of the melting range of the pasty compound is less than or equal to 32° C.

The liquid fraction of the pasty compound measured at 32° C. is equal to the ratio of the enthalpy of fusion consumed at 32° C. to the enthalpy of fusion of the pasty compound. The enthalpy of fusion consumed at 32° C. is calculated in the same manner as the enthalpy of fusion consumed at 23° C.

As used herein, the term “wax” is understood to mean a lipophilic compound that is solid at room temperature (25° C.), with a reversible solid/liquid change of state, having a melting point greater than or equal to 30° C., which may be as high as 120° C. The melting point of the wax may be measured with the aid of a differential scanning calorimeter (D.S.C.), for example the calorimeter sold under the name DSC 30 by the company Mettler.

The waxes may be hydrocarbonaceous, fluorinated and/or silicone-based and may be of plant, mineral, animal and/or synthetic origin. For example, the waxes can have a melting point greater than or equal to 25° C., such as greater than or equal to 45° C.

Among the waxes which may be used in the at least one first composition as disclosed herein, there may be mentioned, by way of non-limiting example, beeswax, Carnauba or Candelilla wax, paraffin, microcrystalline waxes, ceresin or ozokerite; synthetic waxes such as polyethylene or Fischer Tropsch waxes, silicone waxes such as alkyl or alkoxy-dimethicone comprising from 16 to 45 carbon atoms.

The gums are generally high-molecular weight polydimethylsiloxanes (PDMS) or cellulose gums or polysaccharides.

The nature and the quantity of the solid substances depend on the desired mechanical properties and textures. As a guide, the composition may comprise from 0.1% to 50% by weight of waxes, relative to the total weight of the composition, such as from 1% to 30% by weight.

The at least one first composition can thus comprise water, or a mixture of water and at least one hydrophilic organic solvent such as alcohols, for instance linear and branched lower monoalcohols having from 2 to 5 carbon atoms, such as ethanol, isopropanol or n-propanol, and polyols such as glycerine, diglycerin, propylene glycol, sorbitol, penthylene glycol and polyethylene glycols, or alternatively C₂ ethers and hydrophilic C₂-C₄ aldehydes.

The water or the mixture of water and at least one hydrophilic organic solvent may be present in the composition according to the present disclosure in an amount ranging from 0.1% to 99% by weight, relative to the total weight of the composition, such as from 1% to 80%, and from 10% to 80% by weight.

The first composition may comprise, in addition to the graft polymer, at least one additional polymer such as a film-forming polymer.

Among the film-forming polymers that may be used in the at least one first composition of the present disclosure, non-limiting mention may be made of synthetic polymers of the free-radical type or polycondensate type, polymers of natural origin and mixtures thereof. The film-forming polymers may be chosen, by way of non-limiting example, from cellulosic polymers such as nitrocellulose, cellulose acetate, cellulose acetobutyrate, cellulose acetylpropionate, ethyl cellulose or alternatively polyurethanes, acrylic polymers, vinyl polymers, polyvinylbutyrals, alkyd resins, resins derived from the products of condensation of aldehyde such as arylsulphonamide formaldehyde resins such as the toluene sulphonamide formaldehyde resin, the aryl-sulphonamide epoxy resins.

Among the film-forming polymers that may be used as disclosed herein, further non-limiting mention may be made of nitrocellulose RS ⅛ sec; RS ¼ sec; ½ sec; RS 5 sec.; RS 15 sec.; RS 35 sec.; RS 75 sec.; RS 150 sec; AS ¼ sec.; AS ½ sec.; SS ¼ sec.; SS ½ sec.; SS 5 sec., marketed for instance by the company Hercules; the toluene sulphonamide formaldehyde resin “Ketjenfflex MS80” from the company Akzo or “Santolite MHP,” “Santolite MS 80,” from the company Faconnier or “Resimpol 80” from the company Pan Americana, or the alkyd resin “Beckosol ODE 230-70-E” from the company Dainippon, the acrylic resin “Acryloid B66” from the company Rohm & Haas, the polyurethane resin “Trixene PR 4127” from the company Baxenden.

The additional film-forming polymer may be one chosen from the polymers described in French Patent Application No. FR 04/50540, filed on Mar. 18, 2004, the content of which is incorporated herein by reference.

The at least one addition film-forming polymer can be present in the composition according to the present disclosure in an amount ranging from 0.1% to 60% by weight, relative to the total weight of the composition, for instance ranging from 2% to 40% by weight, such as from 5% to 25% by weight.

The at least one first composition may additionally comprise at least one plasticizing agent as described in French Patent Application No. FR 03/14654 filed on Dec. 12, 2003, the content of which is incorporated herein by reference. The quantity of the at least one plasticizer can be chosen by a person skilled in the art on the basis of their general knowledge, so as to obtain a composition having cosmetically acceptable properties.

At Least One Second Composition

The cosmetic product according to the present disclosure comprises at least one second composition comprising a cosmetically acceptable medium.

The second composition can be chosen, for example, such that it improves at least one cosmetic property of the at least one first composition, when the at least one first composition is applied alone to the keratin material. The at least one second composition may improve, for instance, the comfort of the at least one first composition or reduce its sticky character.

The at least one second composition can also be chosen such that the product, once applied to the keratin materials, has satisfactory gloss properties.

The at least one first composition and/or the at least one second composition may be chosen such that the product, once applied to the keratin materials, has satisfactory gloss properties.

Gloss

The mean gloss of the product as disclosed herein, measured at 20°, once the at least one first and the at least one second compositions have been spread one over the other on a support, may be, for example, greater than or equal to 30, for instance greater than or equal to 40, such as greater than or equal to 50, and greater than or equal to 60 out of 100.

As used herein, the expression “mean gloss” is understood to mean the gloss as may be conventionally measured with the aid of a glossmeter by the following method.

It is possible to measure the gloss of a deposit of the at least one first composition alone, the gloss of a deposit of the at least one second composition alone, or the gloss of a deposit of the product according to the present disclosure comprising the superposition of deposits of the at least one first and at least one second compositions.

A coat ranging from 50 μm to 150 μm in thickness of the at least one first and/or at least one second composition is spread using an automatic spreader onto a Leneta brand contrast card of reference Form 1A Penopac. The coat of the at least one first or second composition or of the product covers at least the white background of the card. The deposit is left to dry for 24 hours at a temperature of 30° C., and the gloss at 20° is then measured on the white background using a Byk Gardner brand glossmeter of reference microTri-Gloss. This measurement (ranging from 0 to 100) is repeated at least three times, and the mean gloss is the mean of the at least three measurements taken.

When it is desired to measure the gloss of the product according to the present disclosure, a first coat of one of the at least one first or at least one second composition is spread onto the contrast card, under the same conditions as described previously. This first coat is left to dry for the necessary time, and a second coat of the other composition is then spread on top, under the same conditions as described previously.

The mean gloss of the at least one first composition, or of the at least one second composition, or of the product of the present disclosure, measured at 20° can be, for example, greater than or equal to 30, for instance greater than or equal to 35, such as greater than or equal to 40, and greater than or equal to 45, for instance greater than or equal to 50 out of 100, and greater than or equal to 55, for example greater than or equal to 60, and greater than or equal to 65, such as greater than or equal to 70 or, greater than and equal to 75 out of 100. For certain compositions according to the present disclosure, such as nail varnishes, the gloss measured at 20° may be greater than or equal to 70, or even 80 out of 100.

For example, the mean gloss of the at least one first composition, or of the at least one second composition, or of the product of the present disclosure, once spread onto a support, measured at 60° can be greater than or equal to 50, for instance greater than or equal to 60, and greater than or equal to 65, such as greater than or equal to 70, and greater than or equal to 75, for example greater than or equal to 80, greater than or equal to 85 and, greater than or equal to 90 out of 100.

The measurement of the mean gloss at 60° is performed under the same conditions as those described previously to measure the mean gloss at 20°.

Transfer-Resistance

The at least one first composition and/or the at least one second composition may also be chosen such that the product, once applied to the keratin materials, has satisfactory transfer-resistant properties.

For instance, the at least one first composition can have, for example a transfer index of less than 35, less than or equal to 30, and less than or equal to 20, such as less than or equal to 15, and less than or equal to 10, for instance less than or equal to 5 and less than or equal to 2 out of 100.

The at least one second composition can be chosen such that the transfer index of the product of the present disclosure is less than or equal to 35, such as less than or equal to 30, for instance less than or equal to 20, less than or equal to 15, such as less than or equal to 10 and less than or equal to 5.

The transfer index can be measured according to the following method.

A support (rectangle of 40 mm×70 mm and 3 mm thick) of polyethylene foam that is adhesive on one of its faces, having a density of 33 kg/m³ (sold under the name RE40X70EP3 from the company Joint Technique Lyonnais Ind) is preheated on a hotplate maintained at a temperature of 40° C. in order for the surface of the support to be maintained at a temperature of 33° C.±1° C. The composition is applied over the entire non-adhesive surface of the support, by spreading it using a fine brush to obtain a deposit of about 15 μm of the composition, while leaving the support on the hotplate, and the support is then left to dry for 30 minutes.

The at least one first or at least one second composition is applied over the entire non-adhesive surface of the support, by spreading it using a fine brush to obtain a deposit of about 15 μm of the composition, while leaving the support on the hotplate, and the support is then dried for 30 minutes. After drying, the support is bonded via its adhesive face onto an anvil of diameter 20 mm and equipped with a screw pitch. The support/deposit assembly is then cut up using a punch 18 mm in diameter. The anvil is then screwed onto a press (Statif Manuel Imada SV-2 from the company Someco) equipped with a tensile testing machine (Imada DPS-20 from the company Someco).

White photocopier paper of 80 g/m² is placed on the bed of the press and the support/deposit assembly is then pressed on the paper at a pressure of 2.5 kg for 30 seconds. After removing the support/deposit assembly, some of the deposit has transferred onto the paper. The color of the deposit transferred onto the paper is then measured using a Minolta CR300 calorimeter, the color being characterized by the L*, a*, b* calorimetric parameters. The calorimetric parameters L*₀, a*₀ and b*₀ of the color of the plain paper used is determined.

The difference in color ΔE1 between the color of the deposit transferred relative to the color of the plain paper is then determined according to the following relationship.
ΔE1=\sqrt{(L*−L₀*)²+(a*−a₀*)²+(b*−b₀*)²}

Moreover, a total transfer reference is prepared by applying the at least one first or at least one second composition directly onto a paper identical to the one used previously, at room temperature (25° C.), by spreading the composition using a fine brush and so as to obtain a deposit of about 15 μm of the composition, and the deposit is then left to dry for 30 minutes at room temperature (25° C.). After drying, the colorimetric parameters L*′, a*′ and b*′ of the color of the deposit placed on the paper, corresponding to the reference color of total transfer, are measured directly. The colorimetric parameters L*′₀, a*′₀ and b*′₀ of the color of the plain paper used are determined.

The difference in color ΔE2 between the reference color of total transfer relative to the color of the plain paper are then determined according to the following relationship:
ΔE2=\sqrt{(L*′−L₀*′)²+(a*′−a₀*′)²+(b*′−b₀*′)²}

The transfer of the at least one first or at least one second composition, expressed as a percentage, is equal to the ratio:
100×ΔE1/ΔE2

The measurement is performed on 4 supports in succession and the transfer value corresponds to the mean of the 4 measurements obtained with the 4 supports.

Staying Power

The at least one first composition and/or the at least one second composition can also be chosen such that the product, once applied to the keratin materials, has satisfactory staying power properties. For example, the at least one first composition can have a staying power index greater than or equal to 80%, such as greater than or equal to 85% or greater than or equal to 90%, for instance greater than or equal to 95%. The at least one second composition can be chosen such that the staying power index of the product is greater than or equal to 80%, for example, greater than or equal to 85% or greater than or equal to 90%, such as greater than or equal to 95%.

The staying power of the deposit obtained with the at least one first and/or at least one second compositions according to the present disclosure is determined according to the measurement protocol described below.

A support (rectangle of 40 mm×70 mm) comprising an acrylic coating (hypoallergenic acrylic adhesive on polyethylene film sold under the name Blenderme ref FH5000-55113 by the company 3M Sante) bonded to a layer of foam of adhesive polyethylene on the face opposite that to which the plaster is attached (foam layer sold under the name RE40X70EP3 from the company Joint Technique Lyonnais Ind) is prepared.

The color L*₀a*₀b*₀ of the support, acrylic coating face side, is measured using a Minolta CR 300 calorimeter

The support thus prepared is preheated on a hotplate maintained at the temperature of 40° C. in order for the support to be maintained at a temperature of 33° C.±1° C.

The at least one first and/or at least one second composition is applied over the entire non-adhesive surface of the support (that is to say the surface of the acrylic coating), by spreading it using a fine brush to obtain a deposit of the composition of about 15 μm, while leaving the support on the hotplate, and the support is then left to dry for 10 minutes. After drying, the color L*a*b* of the film thus obtained is measured.

The difference in color ΔE1 between the color of the film relative to the color of the bare support is then determined according to the following relationship.
ΔE1=\sqrt{(L*−L₀*)²+(a*−a₀*)²+(b*−b₀*)²}

The support is then bonded via its adhesive face (adhesive face of the foam layer) onto an anvil of diameter 20 mm and equipped with a screw pitch. A test piece of the support/deposit assembly is then cut up using a punch 18 mm in diameter. The anvil is then screwed onto a press (Statif Manuel Imada SV-2 from the company Someco) equipped with a tensile testing machine (Imada DPS-20 from the company Someco).

A strip 33 mm wide and 29.7 cm long is drawn on a white photocopier paper of weight 80 g/m², a first line is drawn 2 cm from the edge of the sheet, and then a second line 5 cm from the edge of the sheet, the first and second lines thus delimiting a compartment on the strip, and then a first mark and a second mark located in the strip are placed respectively at the reference points 8 cm and 16 cm from the second line. 20 μl of water are placed on the first mark and 10 μl of refined sunflower oil (sold by the company Lesieur) are placed on the second mark.

The white paper is placed on the bed of the press and the test piece placed on the compartment of the paper strip is then pressed at a pressure of about 300 g/cm² exerted for 30 seconds. Next, the press is raised and the test piece is again placed just after the second line (and therefore beside the compartment), a pressure of about 300 g/cm² is again applied and the paper is moved, rectilinearly as soon as contact is made, at a speed of 1 cm/s, over the entire length of the strip such that the test piece crosses the deposits of water and oil.

After removing the test piece, a portion of the deposit was transferred onto the paper. The color L*′,a*′,b*′ of the deposit remaining on the test piece is then measured. The difference in color ΔE2 between the color of the deposit remaining on the test piece relative to the color of the bare support is then determined by the following relationship.
ΔE2=\sqrt{(L*′−L₀*′)²+(a*′−a₀*)²+(b*′−b₀*)²}

The staying power index of the composition, expressed as a percentage, is equal to the ratio:
100×ΔE1/ΔE2

The measurement is performed on 6 supports in succession and the transfer value corresponds to the mean of the 6 measurements obtained with the 6 supports.

The cosmetically acceptable medium of the at least one second composition can comprise, for example, a liquid phase that is non-volatile at room temperature and atmospheric pressure.

As used herein, the expression “non-volatile liquid phase” is understood to mean any medium capable of remaining on the skin or the lips for several hours. A non-volatile liquid phase has, for instance, a non-zero vapor pressure at room temperature and at atmospheric pressure of less than 0.02 mmHg (2.66 Pa) such as less than 10⁻³ mmHg (0.13 Pa).

The non-volatile liquid phase of the at least one second composition can be at least one phase chosen from hydrocarbon phases, one liquid silicone phases, and fluorinated phases that are liquid at room temperature.

As used herein, the expression “non-volatile liquid hydrocarbon phase” is understood to mean a phase comprising at least one oil comprising predominantly carbon atoms and hydrogen atoms, for instance, alkyl or alkenyl chains such as alkanes or alkenes, and also oils with an alkyl or alkenyl chain comprising at least one group chosen from ether, ester or carboxylic acid groups.

The non-volatile liquid phase of the at least one second composition can be present in an amount, for example, ranging from 1% to 100%, for instance ranging from 5% to 95%, such as from 20% to 80% and from 40% to 80% by weight, relative to the total weight of the at least one second composition.

Non-Volatile Silicone Oil of the Liquid Phase

The non-volatile liquid phase of the at least one second composition can comprise, for instance, at least one silicone oil, for example a phenylsilicone oil or a polydimethylsiloxane oil. The non-volatile silicone oil can be chosen from polyalkylsiloxanes, polyarylsiloxanes and polyalkylarylsiloxanes, and mixtures thereof.

The non-volatile silicone oil may be chosen from, by way of non-limiting examples:
linear or branched non-volatile polydimethylsiloxanes (PDMS);
polydimethylsiloxanes comprising alkyl, alkoxy or phenyl groups, which are pendent or at the end of a silicone chain, these groups comprising from 2 to 24 carbon atoms;
phenylsilicones, for instance phenyl trimethicones, phenyl dimethicones, phenyltrimethylsiloxydiphenylsiloxanes, diphenyl dimethicones, diphenylmethyl-diphenyltrisiloxanes and 2-phenylethyl trimethylsiloxysilicates.

Among the polyalkylsiloxanes that may be used according to the present disclosure, non-limiting mention may be made of polydimethylsiloxanes, (polydimethylsiloxane) (methylvinylsiloxane) copolymers, poly(dimethyl-siloxane)(diphenyl)siloxanes, poly(dimethylsiloxane)(diphenyl)(methylvinylsiloxane) copolymers, and mixtures thereof.

In one embodiment of the present disclosure, the non-volatile silicone oil can be chosen from the silicones of formula (II): 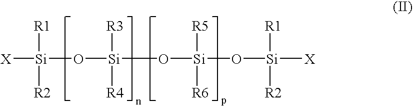
wherein:

R₁, R₂, R₅ and R₆, which may be identical or different, are chosen from alkyl radicals comprising from 1 to 6 carbon atoms,

R₃ and R₄, which may be identical or different, are chosen from alkyl radicals comprising from 1 to 6 carbon atoms or an aryl radical,

X is chosen from alkyl radicals comprising from 1 to 6 carbon atoms, hydroxyl radicals and vinyl radicals,

n and p are chosen so as to give the oil a weight-average molecular mass of less than 200,000 g/mol, for instance, less than 150,000 g/mol, such as less than 100 000 g/mol.

As an example of a non-volatile silicone oil of formula (II), non-limiting mention may be made of polydimethylsiloxane with a viscosity ranging from 0.5 cSt to 60,000 cSt, for instance ranging from 0.5 cSt to 10,000 cSt, such as from 0.5 cSt to 1000 cSt measured according to ASTM standard D-445, for example DC 200 of viscosity 350 cSt sold by Dow Corning.

According to one embodiment of the present disclosure, the at least one second composition comprises a non-volatile silicone oil of formula (II) such as a polydimethylsiloxane with a viscosity ranging, for instance, from 0.5 cSt to 500 cSt, such as ranging from 1 cSt to 10 cSt, for example the polydimethylsiloxane sold under the name DC 200 of viscosity 5 cSt and of molecular weight 800, sold by Dow Corning.

The weight-average molecular mass of the non-volatile silicone oil can, for example, range from 400 to 200,000, such as from 4,000 to 100,000, from 4,000 to 20,000, for instance from 400 to 2,000 and from 400 to 1,000 g/mol.

Non-Volatile Fluorinated Oil of the Liquid Phase

The non-volatile liquid phase of the at least one second composition can comprise at least one fluorinated oil of formula (III): 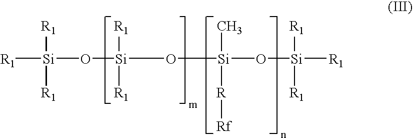
wherein:
R is chosen from linear and branched alkylenyl groups comprising from 1 to 6 carbon atoms, for instance a divalent methylenyl, ethylenyl, propylenyl or butylenyl group,
Rf is chosen from fluoroalkyl radicals, such as perfluoroalkyl radical, comprising from 1 to 9 carbon atoms, such as from 1 to 4 carbon atoms,
R₁, which may be identical or different, are chosen from C₁-C₂₀, such as C₁-C₄, alkyl radicals; hydroxyl radicals; and phenyl radicals,
m ranges from 0 to 150, such as from 20 to 100, and
n ranges from 1 to 300, such as from 1 to 100.

For example, in one embodiment of the present disclosure, the oils of formula (III) are such that R1 is a methyl, R is an ethyl and Rf is CF3. Among the fluorosilicone compounds of formula (III) that may be mentioned, in a non-limiting manner, are those sold by the company Shin Etsu under the names “X22-819”, “X22-820”, “X22-821” and “X22-822” or “FL-100”.

Further among the fluorinated oils that may also be mentioned, in a non-limiting manner, are the fluorinated polyethers chosen from the compounds of formula (IV):
R₆—(CF₂—CFR₃—CF₂O)p-(CFR₄—CF₂—O)q-(CFR₅—O)r-R₇ (IV)
wherein:
R₃ to R₆, which may be identical or different, are chosen from monovalent radicals chosen from —F, —(CF₂)ₙ—CF₃ and —O—(CF₂)ₙ—CF₃,
R₇ is a monovalent radical chosen from —F and —(CF₂)ₙ—CF₃,
with n ranging from 0 to 4,
p ranging from 0 to 600, q ranging from 0 to 860, r ranging from 0 to 1500, and p, q and r being integers chosen such that the weight-average molecular mass of the compound ranges from 500 to 100,000, for instance from 500 to 10,000.

The fluorinated oils may also be chosen from fluoroalkanes chosen from C₂-C₅₀, such as from C₅-C₃₀ perfluoroalkanes and fluoroalkanes, such as perfluorodecalin, perfluoroadamantane and bromoperfluorooctyl, and mixtures thereof.

Volatile Oil of the Liquid Phase

The at least one second composition may optionally comprise a volatile oil. These oils may be hydrocarbon-based oils or silicone oils optionally comprising alkyl or alkoxy groups that are pendent or at the end of a silicone chain, or a mixture of these oils.

For example, the volatile oils can be cosmetic oils chosen from oils with a flash point ranging from 40° C. to 100° C., and mixtures thereof. In addition, they can have, for instance, a boiling point at atmospheric pressure of less than 220° C., such as less than 210° C., for instance ranging from 110° C. to 210° C. In one embodiment of the present disclosure, the volatile oils are not monoalcohols comprising at least 7 carbon atoms.

Among the volatile oils that may be used in the present disclosure, non-limiting mention may be made of linear or cyclic silicone oils with a viscosity at room temperature of less than 8 cSt, for instance comprising from 2 to 7 silicon atoms, these silicones optionally comprising alkyl or alkoxy groups of 1 to 10 carbon atoms. Further among volatile silicone oils that may be used in the present disclosure, non-limiting mention may also be made of octamethylcyclotetrasiloxane, decamethylcyclopentasiloxane, dodecamethylcyclohexasiloxane, heptamethylhexyltrisiloxane, heptamethyloctyltrisiloxane, hexamethyldisiloxane, octamethyltrisiloxane, decamethyltetrasiloxane and dodecamethylpentasiloxane, and mixtures thereof.

As non-limiting examples of other volatile oils that may be used in the present disclosure, mention may be made of hydrocarbon-based volatile oils comprising from 8 to 16 carbon atoms and mixtures thereof, and such as C₈-C₁₆ branched alkanes, for instance C₈-C₁₆ isoalkanes (also known as isoparaffins), isododecane, isodecane and isohexadecane, and for example the oils sold under the trade names Isopar or Permethyl, and C₈-C₁₆ branched esters, for instance isohexyl neopentanoate, and mixtures thereof.

In one embodiment of the present disclosure, isododecane (Permethyl 99 A), C₈-C₁₆ isoparaffins, for instance Isopar L, E, G or H, and mixtures thereof, optionally combined with decamethyltetrasiloxane or with cyclopentasiloxane, are used.

In general, the amount of volatile oil that is used is an amount that is sufficient to improve the spreading qualities of the at least one second composition. This amount will be adapted by a person skilled in the art as a function of the intensity of the desired properties.

The amount of volatile oil is chosen so that it does not reduce the gloss of the at least one second composition. According to another embodiment of the present disclosure, the second composition does not comprise a volatile oil.

High Molecular Weight Polymer

The at least one second composition can comprise, for example, a high molecular weight polymer that is different from the non-volatile silicone oil described above.

When the at least one second composition according to the present disclosure is liquid, it can comprise, for example, from 20% to 50% by weight of a high molecular weight polymer.

According to one embodiment of the present disclosure, the second composition can comprise, for example, the mixture of a polydimethylsiloxane having a molecular mass ranging from 200,000 to 300,000 g/mol and a polydimethylsiloxane having a molecular mass ranging from 400 to 1,000 g/mol.

The mass ratio of the high molecular weight silicone compound/liquid silicone compound can range, for example, from 20/80 to 60/40, such as from 35/65 to 45/55.

When the at least one second composition according to the present disclosure is solid, it can comprise, for example, from 2% to 40% by weight of a high molecular weight polymer, relative to the total weight of the composition.

In one embodiment of the present disclosure, the polymer is a silicone polymer.

The polymer can be liquid or solid at room temperature and its weight-average molecular mass is greater than or equal to 200,000 g/mol, such as from 200,000 and 2,500,000, for instance from 200,000 to 2,000,000 g/mol. When the polymer is liquid, it is part of the non-volatile liquid phase of the at least one second composition.

The viscosity of the polymer can range from 10,000 to 5,000,000 cSt, for instance from 100,000 to 1,000,000 cSt, such as from 300,000 to 700,000 cSt, measured according to ASTM standard D-445.

The high molecular weight polymer can be, for example, an ungrafted polymer, i.e. a polymer obtained by polymerizing at least one monomer, without subsequent reaction of the side chains with another chemical compound. The polymer can be chosen from, for instance, dimethiconols, fluorosilicones, dimethicones and mixtures thereof. The polymer can be, for example, a homopolymer.

In one embodiment of the present disclosure, for example, the polymer that can be used is a high molecular weight polymer chosen from those of formula (V): 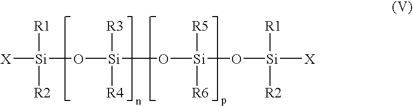
wherein:

R₁, R₂, R₅ and R₆, which may be identical or different, are chosen from alkyl radicals comprising from 1 to 6 carbon atoms, optionally substituted with at least one fluorine atom,

R₃ and R₄, which may be identical or different, are chosen from alkyl radicals comprising from 1 to 6 carbon atoms, and aryl radicals,

X is chosen from alkyl radicals comprising from 1 to 6 carbon atoms, hydroxyl radicals, vinyl and allyl radicals, and alkoxy radicals comprising from 1 to 6 carbon atoms, and

n and p are chosen such that the silicone compound has a weight-average molecular mass of greater than or equal to 200,000 g/mol.

For example, p may be equal to 0.

In one embodiment of the present disclosure, the polymers of formula (V) are such that R₁ to R₆ are methyl groups and the substituent X is a hydroxyl group, i.e., they are dimethiconols. Further non-limiting examples that can be mentioned include the polymers of formula (V) such that p=0 and n ranges from 2,000 to 40,000, such as from 3,000 to 30,000. Non-limiting mention may also be made of polymers with a molecular mass ranging from 1,500,000 to 2,000,000 g/mol.

According to another embodiment of the present disclosure, the high molecular weight polymer is the dimethiconol sold by Dow Corning in a polydimethylsiloxane (5 cSt) under the reference D2-9085, the viscosity of the mixture being equal to 1550 cSt, or the dimethiconol sold by Dow Corning in a polydimethylsiloxane (5 cSt) under the reference DC 1503. The dimethiconol (of molecular weight equal to 1,770,000 g/mol) sold by Dow Corning under the reference Q2-1403 or Q2-1401, the viscosity of the mixture being equal to 4000 cSt, can also be used.

Among the high molecular weight polymers of formula (V) that may be used according to the present disclosure, non-limiting mention may be made of those for which:
the substituents R₁ to R₆ and X are methyl groups, for instance the product sold under the name SE30 by the company General Electric, and the product sold under the name AK 500000 by the company Wacker,
the substituents R₁ to R₆ and X are methyl groups and p and n are such that the molecular weight is 250,000 g/mol, for instance the product sold under the name Silbione 70047 V by the company Rhodia,
the substituents R₁ to R₆ are methyl groups and the substituent X is a hydroxyl group, for instance the product sold under the name Q2-1401 or Q2-1403 by the company Dow Corning,
the substituents R₁, R₂, R₅, R₆ and X are methyl groups, the substituents R₃ and R₄ are aryl groups and n and p are such that the molecular weight of the polymer is 600,000 g/mol, for instance the product sold under the name 761 by the company Rhône-Poulenc.

The high molecular weight silicone polymer can be, for example, introduced into the composition in the form of a mixture with a liquid silicone, the viscosity of the liquid silicone ranging from 0.5 cSt to 10,000 cSt, such as from 0.5 cSt to 500 cSt, for instance, from 1 cSt to 10 cSt.

The fluid silicone may be chosen from polyalkylsiloxanes, polyarylsiloxanes, polyalkylarylsiloxanes and mixtures thereof. The liquid silicone may be a volatile silicone such as a cyclic polydimethylsiloxane comprising from 3 to 7 —(CH₃)₂SiO— units.

The liquid silicone may also be a non-volatile polydimethylsiloxane silicone, for instance, with a viscosity ranging from 0.5 cSt to 10,000 cSt, such as 5 cSt, for example the silicone sold under the reference DC 200 by Dow Corning.

The ratio of the high molecular weight silicone polymer in the mixture of the high molecular weight silicone polymer and liquid silicone is can range from 10/90 to 20/80. The viscosity of the mixture of the high molecular weight silicone polymer and liquid silicone can range, for example, from 1,000 to 10,000 cSt.

Non-limiting examples of the high molecular weight dimethicones according to the present disclosure, include the dimethicones described in U.S. Pat. No. 4,152,416. They are sold, for example, under the references SE30, SE33, SE54 and SE76.

The dimethicones according to the present disclosure are, for example, compounds of formula (III) such that R₁ to R₆ and X are methyl groups and p is equal to 0. The molecular weight of these polymers can range, for example, from 200,000 to 300,000, such as from 240,000 to 260,000 g/mol.

Non-limiting examples of the dimethicones according to the present disclosure include polydimethylsiloxanes, (polydimethylsiloxane) (methylvinylsiloxane) copolymers and poly(dimethylsiloxane)(diphenyl)(methylvinylsiloxane) copolymers, and mixtures thereof.

The high molecular weight fluorosilicones according to the present disclosure can have for instance, a molecular weight ranging from 200,000 to 300,000, such as from 240,000 to 260,000 g/mol.

According to one aspect of the present disclosure, the at least one second composition according to the present disclosure comprises at least one apolar or sparingly polar compound, which may be chosen from oils, gums and/or waxes. The at least one second composition can comprise for example, more than 70%, such as more than 80% by weight, and for instance, 100% by weight of apolar or sparingly polar compounds. These apolar or sparingly polar compounds comprise coloring agents or gelling agents.

According to one embodiment of the present disclosure, the at least one second composition comprises the mixture of a polymer with a weight-average molecular mass of greater than or equal to 200,000 g/mol and a silicone oil as described above.

According to another embodiment of the present disclosure, the at least one second composition is transparent. As used herein, the term “transparent composition” means a transparent to translucent composition, i.e. a composition which is such that it transmits at least 40% of light such as at least 50% of light at a wavelength of 750 nm.

The transmission is measured using a Cary 300 Scan UV-visible spectrophotometer from the company Varian, according to the following protocol: The composition is poured above its melting point into a spectrophotometer cuvette of square cross section with a side length of 10 mm. The sample of the composition is then cooled for 24 hours at 35° C., and then kept in a chamber thermostatically maintained at 20° C. for 24 hours. The light transmitted through the sample of the composition is then measured by spectrophotometer by scanning wavelengths ranging from 700 nm to 800 nm, the measurement being performed in transmission mode. The percentage of light transmitted through the sample of the composition at a wavelength of 750 nm is then determined.

When the at least one second composition is transparent, it comprises, for example, less than 5%, such as less than 2%, and for instance less than 1% of pigments.

The mixture of high molecular weight polymer and liquid silicone compound can be present, for example, in an amount greater than or equal to 70%, such as greater than or equal to 80% by weight, greater than or equal to 90% by weight and greater than or equal to 95% by weight, relative to the weight of the at least one second composition.

The at least one second composition may comprise other compounds, which can be, for example, apolar or sparingly polar. These apolar or sparingly polar compounds can be silicone compounds, coloring agents or gelling agents.

According to one embodiment of the present disclosure, the at least one second composition comprises only apolar or sparingly polar ingredients.

The at least one second composition can comprise at least one wax, such as when it is in solid form. The at least one wax can be present in an amount ranging from 0.5% to 30% by weight, relative to the total weight of the composition, for instance from 5% to 20%, such as from 5% to 15% by weight, relative to the total weight of the composition.

Linear hydrocarbon-based waxes, for example, can be used as disclosed herein. Their melting point can be for instance, greater than or equal to 35° C., for example greater than 55° C., such as greater than 80° C.

The linear hydrocarbon-based waxes can be chosen from, for example, substituted linear alkanes, unsubstituted linear alkanes, unsubstituted linear alkenes and substituted linear alkenes, an unsubstituted compound being composed exclusively of carbon and hydrogen. The substituents mentioned above do not contain carbon atoms.

The linear hydrocarbon-based waxes can include ethylene polymers and copolymers with a molecular weight ranging from 400 and 800, for example Polywax 500 or Polywax 400 sold by New Phase Technologies.

The linear hydrocarbon-based waxes can include linear paraffin waxes, for instance the paraffins S&P 206, S&P 173 and S&P 434 from Strahl & Pitsch.

The linear hydrocarbon-based waxes can also include long-chain linear alcohols, for instance products comprising a mixture of polyethylene and of alcohols comprising from 20 to 50 carbon atoms, such as Performacol 425 or Performacol 550 (mixture in 20/80 proportions) sold by New Phase Technologies.

The at least one second composition can comprise, for example, a silicone wax, such as a dimethicone comprising alkyl groups at the end of a chain. These alkyl groups can comprise, for instance, more than 18 carbon atoms, such as ranging from 20 to 50, for example, from 30 to 45 carbon atoms.

The silicone wax can be chosen from those of formulae (VI) and (VII) 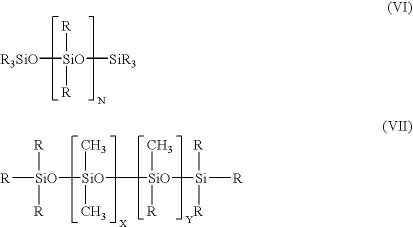
wherein R is an alkyl, X is greater than or equal to zero and N and Y are greater than or equal to one.

R comprises from 1 to 50 carbon atoms provided that the compound is solid at room temperature.

Non-limiting examples of silicone waxes include:
C₂₀₋₂₄ alkyl methicone, C₂₄₋₂₈ alkyl dimethicone, C₂₀₋₂₄ alkyl dimethicone and C₂₄₋₂₈ alkyl dimethicone sold by Archimica Fine Chemicals under the reference SilCare 41 M40, SilCare 41 M50, SilCare 41 M70 and SilCare 41 M80,
the stearyl dimethicones of reference SilCare 41 M65 sold by Archimica or of reference DC-2503 sold by Dow Corning,
the stearoxytrimethylsilanes sold under the reference SilCare 1M71 or DC-580, the products Abil Wax 9810, 9800 or 2440 from Wacker-Chemie GmbH,
the C₃₀₋₄₅ alkyl methicones sold by Dow Corning under the reference AMS-C30 Wax, and also the C₃₀₋₄₅ alkyldimethicones sold by General Electric under the reference SF 1642 or SF 1632.

When the at least one first and the at least one second compositions are intended to be applied to the nails, the at least one first and/or at least one second composition can comprise, for example, a solvent chosen from:
ketones that are liquid at room temperature, such as methyl ethyl ketone, methyl isobutyl ketone, diisobutyl ketone, isophorone, cyclohexanone or acetone;
alcohols that are liquid at room temperature, such as ethanol, isopropanol, diacetone alcohol, 2-butoxyethanol or cyclohexanol;
glycols that are liquid at room temperature, such as ethylene glycol, propylene glycol, pentylene glycol or glycerol;
propylene glycol ethers that are liquid at room temperature, such as propylene glycol monomethyl ether, propylene glycol monomethyl ether acetate or dipropylene glycol mono n-butyl ether;
cyclic ethers such as γ-butyrolactone;
short-chain esters (comprising from 3 to 8 carbon atoms in total) such as ethyl acetate, methyl acetate, propyl acetate, isopropyl acetate, n-butyl acetate, isopentyl acetate, methoxypropyl acetate or butyl lactate;
ethers that are liquid at room temperature, such as diethyl ether, dimethyl ether or dichlorodiethyl ether;
alkanes that are liquid at room temperature, such as decane, heptane, dodecane or cyclohexane;
alkyl sulphoxides such as dimethyl sulphoxide;
aldehydes that are liquid at room temperature, such as benzaldehyde or acetaldehyde;
heterocyclic compounds such as tetrahydrofuran;
propylene carbonate or ethyl 3-ethoxypropionate;
mixtures thereof.

In one embodiment of the present disclosure, the solvent is chosen from methyl acetate, isopropyl acetate, methoxypropyl acetate, butyl lactate, acetone, methyl ethyl ketone, diacetone alcohol, γ-butyrolactone, tetrahydrofuran, propylene carbonate, ethyl 3-ethoxypropionate and dimethyl sulphoxide, and mixtures thereof.

The at least one first and/or at least one second composition of the cosmetic product according to the present disclosure may comprise at least one coloring agent which can be chosen from water-soluble or fat-soluble colorants, from pigments, from pearlescent agents and from mixtures thereof.

As used herein, the term “pigments” is understood to mean white or colored, inorganic or organic particles that are insoluble in the liquid organic phase, intended to color and/or opacify the first composition.

As used herein, the expression “pearlescent agents” is understood to mean iridescent particles produced for instance, by certain molluscs in their shell or alternatively synthesized, which are insoluble in the medium of the first composition.

As used herein, the term “colorants” is understood to mean generally organic compounds that are soluble in fatty substances such as oils or in an aqueous-alcoholic phase.

The at least one first and/or at least one second composition according to the present disclosure may additionally comprise at least one coloring material chosen from water-soluble colorants, and pulverulent coloring materials such as pigments, pearlescent agents, and glitter which is known to persons skilled in the art. The at least one coloring material can be present in the at least one first and/or at least one second composition, when present, in an amount ranging from 0.01% to 50% by weight, relative to the weight of the composition, such as from 0.01% to 30% by weight.

The solid pigments, pearlescent agents or fillers may be dispersed in the liquid fatty phase of the composition in the presence of at least one dispersing agent. The at least one dispersing agent can serve to protect the dispersed particles against their agglomeration or flocculation. The dispersing agent may be a surfactant, an oligomer, a polymer or a mixture of several of them, bearing one or more functionalities having a high affinity for the surface of the particles to be dispersed. For instance, they may become physically or chemically attached to the surface of the pigments. These dispersants additionally have at least one functional group that is compatible or soluble in the continuous medium. By way of non-limiting example, use may be made of the esters of 12-hydroxystearic acid such as of a C₈ to C₂₀ fatty acid and a polyol such as glycerol, diglycerin, such as the stearate of poly(12-hydroxystearic) acid having a molecular weight of 750 g/mol such as that sold under the name Solsperse 21 000 by the company Avecia, polyglycerol-2 dipolyhydroxystearate (CTFA name) sold under the reference Dehymyls PGPH by the company Henkel or polyhydroxystearic acid such as that sold under the reference Arlacel P100 by the company Uniqema, and mixtures thereof.

Among the other dispersants which can be used in the at least one first and/or at least one second composition of the present disclosure, non-limiting mention may be made of quaternary ammonium derivatives of polycondensed fatty acids such as Solsperse 17 000 sold by the company Avecia, mixtures of poly dimethylsiloxane/oxypropylene such as those sold by the company Dow Corning under the references DC2-5185, DC2-5225 C.

The polyhydroxystearic acid and the esters of 12-hydroxystearic acid can be used, for example, for a hydrocarbon-based or fluorinated medium, while the mixtures of dimethylsiloxane oxyethylene/oxpropylene can be used, for instance, for a silicone medium.

The pigments may be white or colored, inorganic and/or organic. There may be mentioned, by way of non-limiting example among the inorganic pigments, titanium dioxide, optionally surface-treated, zirconium or cerium oxides, and zinc, iron (black, yellow or red) or chromium oxides, manganese violet, ultramarine blue, chromium hydrate and ferric blue, metallic powders such as aluminium powder and copper powder.

Among the organic pigments, there may be mentioned by way of non-limiting example, carbon black, pigments of the D & C type, and lacquers based on carmine, barium, strontium, calcium or aluminium.

Non-limiting mention may also be made of pigments with effect such as particles comprising a natural or synthetic, organic or inorganic substrate, for example glass, acrylic resins, polyester, polyurethane, polyethylene terephthalate, ceramics or aluminas, the substrate being coated or not with metallic substances such as aluminium, gold, silver, platinum, copper, bronze, or metallic oxides such as titanium dioxide, iron oxide, chromium oxide and mixtures thereof.

The pearlescent pigments may be chosen from mica coated with titanium or bismuth oxychloride, mica-titanium coated with iron oxides, mica-titanium coated with for instance, ferric blue or chromium oxide, mica-titanium coated with an organic pigment of the abovementioned type and pearlescent pigments based on bismuth oxychloride. It is also possible to use interferential pigments, such as those comprising liquid crystals or multilayers.

The water-soluble colorants can be, by way of non-limiting example, beet juice or methylene blue.

The composition according to the present disclosure may comprise at least one filler, for example, in an amount ranging from 0.01% to 50% by weight, relative to the total weight of the composition, such as ranging from 0.01% to 30% by weight. As used herein, the term “fillers” is understood to mean particles of any shape, that are colorless or white, inorganic or synthetic, insoluble in the composition medium regardless of the temperature at which the composition is manufactured. The fillers can serve for instance to modify the rheology or the texture of the composition.

The fillers may be inorganic or organic, of any shape, platelet, spherical or oblong, regardless of the crystallographic shape (for example sheet, cubic, hexagonal, orthorhombic and the like). There may be mentioned, by way of non-limiting example, talc, mica, silica, kaolin, polyamide powder (Nylon®) (Orgasol® from Atochem), poly-β-alanine powder and polyethylene powder, powders of tetrafluoroethylene polymers (Teflon®, lauroyllysine, starch, boron nitride, polymeric hollow microspheres such as those of polyvinylidene chloride/acrylonitrile such as Expancel® (Nobel Industrie), copolymers of acrylic acid (Polytrap® from the company Dow Corning) and microbeads of silicone resin (Tospearls® from Toshiba, for example), particles of elastomeric polyorganosiloxanes, precipitated calcium carbonate, magnesium carbonate and hydrocarbonate, hydroxyapatite, hollow silica microspheres (Silica Beads® from Maprecos), glass or ceramic microcapsules, metallic soaps derived from organic carboxylic acids comprising from 8 to 22 carbon atoms, such as from 12 to 18 carbon atoms, for example zinc, magnesium or lithium stearate, zinc laurate or magnesium myristate.

The at least one first and/or the at least one second composition of the present disclosure may also comprise at least one cosmetic or dermatological active agents such as those conventionally used.

Among the cosmetic, dermatological, hygiene or pharmaceutical active agents that may be used in the at least one first and/or at least one second compositions of the present disclosure, non-limiting mention may be made of moisturizers, vitamins, essential fatty acids, sphingolipids and sunscreens. These active agents are used in the usual amount for those skilled in the art, such as in an amount ranging from 0% to 20%, for instance from 0.001% to 15% by weight, relative to the total weight of the at least one first and/or at least one second composition.

The at least one first and/or at least one second composition may also comprise at least one other additive usually used in such compositions, such as water, antioxidants, fragrances, preserving agents and essential oils.

Needless to say, a person skilled in the art will take care to select this or these optional additional compounds, and/or the amount thereof, such that the beneficial properties of the at least one first and/or at least one second composition are not, or are not substantially, adversely affected by the envisaged addition.

The at least one first and the at least one second compositions of the product may be in the form of a cast product, for example in the form of a stick or wand, or in the form of a dish that may be used by direct contact or with a sponge. For instance, they find an application as cast foundations, cast blushers or eyeshadows, lipsticks, lipcare bases or lipcare balms, concealer products and nail varnishes. They may also be in the form of a soft paste or a gel, a more or less fluid cream, or a liquid packaged in a tube.

The at least one first and at least one second compositions of the product according to the present disclosure may be in the form of, for example, a cosmetic care composition for the face, the neck, the hands or the body (for example a care cream, an antisun oil or a body gel), a makeup composition (for example a makeup gel, a cream or a stick) or a composition for artificially tanning or protecting the skin.

The at least one first and at least one second compositions of the product according to the present disclosure may be in the form of a care composition for the skin and/or the superficial body growths or in the form of an antisun composition or a body hygiene composition, such as in the form of a deodorant. If such forms are the case, then they may be, for instance, in uncolored form. They may then be used as a care base for the skin, the superficial body growths or the lips (lip balms, for protecting the lips against the cold and/or sunlight and/or the wind, or care creams for the skin, the nails or the hair).

As used herein, the term “cosmetically acceptable” is understood to mean a composition of pleasant appearance, smell and feel.

Each composition of the product according to the present disclosure may be in any galenical form normally used for topical application, for instance in the form of an oily or aqueous solution, an oily or aqueous gel, an oil-in-water or water-in-oil emulsion, a multiple emulsion, a dispersion of oil in water using vesicles, the vesicles being located at the oil/water interface, or a powder. Each composition may be fluid or solid.

According to one embodiment of the present disclosure, the at least one first and/or the at least one second composition, can have a continuous fatty phase for instance, in anhydrous form and may comprise less than 5% water, such as less than 1% water relative to the total weight of the at least one first and/or at least one second composition.

Each first and second composition may have the appearance of a lotion, a cream, an ointment, a soft paste, a salve, a solid that has been cast or moulded, such as a stick or a dish, or a compacted solid. Each composition may be packaged separately in the same packaging article, for example in a two-compartment pen, the base composition being delivered from one end of the pen and the top composition being delivered from the other end of the pen, each end being closed, for example, in a leaktight manner with a lid.

Alternatively, each of the compositions may be packaged in a different packaging article.

For example, the composition that is applied as the first coat can be in liquid or pasty form, which can be desirable in the case of a lipstick or an eyeliner.

The product according to the present disclosure may be used, for example, for making up the skin and/or the lips and/or the superficial body growths depending on the nature of the ingredients used. For instance, the product of the present disclosure may be in the form of a solid foundation, a lipstick wand or paste, a concealer product, an eye contour product, an eyeliner, a mascara, an eyeshadow, a body-makeup product or a skin-coloring product.

In one embodiment of the present disclosure, the at least one second composition has care, gloss or transparency properties.

The present disclosure relates to a lip product, a varnish, a mascara, a foundation, a tattoo, a makeup rouge or an eyeshadow comprising at least one first and at least one second compositions as described above.

The compositions of the product of the present disclosure may be obtained by heating the various constituents to the melting point of the highest-melting waxes, followed by casting the molten mixture in a mould (dish or finger stall). They may also be obtained by extrusion, as described in European Patent Application No. EP-A-0 667 146.

Other than in the operating examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present disclosure. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the disclosure are approximations, the numerical values set forth in the specific examples are reported as precisely as possible. Any numerical value, however, inherently contain certain errors necessarily resulting from the standard deviation found in their respective testing measurements.

The following examples are intended to illustrate the present disclosure in a non-limiting manner.

### EXAMPLES

### Example 1

### Polymer Obtained by Polymerization of Methyl Acrylate and the Macromonomer Corresponding to a Polyethylene/Polybutylene Copolymer with a Methacrylate End Group (Kraton L-1253)

2 kg of heptane, 2 kg of isododecane, 2.8 kg of methyl acrylate and 1.2 kg of macromonomer of the polyethylene/polybutylene type with a methacrylate end group (Kraton L-1253) and 320 g of tert-butyl peroxy-2-ethylhexanoate (Trigonox 21S) were loaded into a reactor.

The reaction mixture was stirred and heated at room temperature to 90° C. in 1 hour. After 15 minutes at 90° C., a change was observed in the appearance of the reaction medium, which passed from a transparent appearance to a milky appearance. The heating was maintained, with stirring, for an additional 15 minutes and then a mixture comprising 16 kg of methyl acrylate and 200 g of Trigonox 21S was added dropwise over 1 hour.

The heating was then maintained for 4 hours at 90° C. and then the heptane was distilled off from the reaction medium. At the end of this distillation operation, a dispersion of particles of polymer thus prepared was obtained that was stable in isododecane.

The graft polymer comprised 6% by weight of macromonomer relative to the weight of the polymer.

The characteristics of the polymer and of the particles formed by the polymer were as follows:
weight-average molecular mass Mw=119,900
number-average molecular mass Mn=16,300
polydispersity value (Mw/Mn)=7.37
glass transition: 10° C. by DSC Mettler;
dry extract: 52.4% in isododecane, performed using a thermobalance;
particle size: 46 nm with a polydispersity of 0.05 performed on a Malvern Autosizer Lo-C at 25° C.

The stability of the dispersion obtained was demonstrated by the use of the following stability protocol: 8 ml of the dispersion produced were placed in a haemolysis tube and centrifuged at 4000 revolutions/min for 15 minutes with the aid of a Jouan C100-S5 centrifuge. After 15 minutes, no phase separation was observed therein, demonstrating that the dispersion was stable.

### Example 2

First Composition: Lipstick

The waxes, the pigment pastes and the sucrose ester were introduced into a pan, with Rayneri stirring, the temperature was 105° C., the mixture was kept stirring for 30 minutes and then the pearlescent agents were added, and the polymer dispersion and the fragrance were then added, the mixture was kept stirring for 10 minutes, and then poured into a mold at 42° C. The mold was placed in a freezer and the mixture was unmolded when the mold was at about 4° C.

| Ingredients | % by weight |
|---|---|
| Pigments | 8.20 |
| Hydrogenated polyisobutene (Parleam) | 5.18 |
| Polyhydroxystearic acid (Octacare DSPOL300, Avecia) | 0.21 |
| C30-C50 alcohols (Performacol 550 Alcohol, New | 2 |
| Phase Technologies) | |
| Polyethylene (Polywax 500, Bareco) | 10 |
| Sucrose acetate isobutyrate (Eastman SAIB | 5 |
| Special, Eastman Chemical) | |
| Polymer of Example 1 | 68.82 |
| Fragrance | qs |

Second Composition: Colorless Lipstick

| Ingredients | % by weight |
|---|---|
| Silicone oil (PDMS) | 25 |
| DC200 from Dow Corning (5 cSt) | |
| DIMETHICONE (and) DIMETHICONOL | 61 |
| D2-9085 from Dow Corning (1550 cSt) | |
| TRIFLUOROPROPYL DIMETHICONE (100 cSt) | 1 |
| X22-819 from Shin Etsu | |
| C30-45 ALKYL DIMETHICONE | 5 |
| (SF 1642 from GE Bayer Silicone) | |
| POLYETHYLENE wax (weight-average MW 500) | 8 |

The silicone oil, the dimethiconol and the fluorinated dimethicone were mixed in the hot state until a homogeneous mixture was formed. The C₃₀-C₄₅ alkyl dimethicone was then added to the preceding mixture heated to 110° C. The polyethylene wax was then added little by little until a homogeneous mixture was obtained. The mixture was cooled to 90-95° C. and then poured into the moulds, which were placed at −20° C. for thirty minutes. The sticks were finally unmolded.

The staying power during a meal of the make-up product for the lips comprising the two compositions described above was measured according to the method described above. The value was 98.95 (measurement error 0.48).

The gloss of the make-up product for the lips, measured according to the method described above, was equal to 129.8.

The transfer of the make-up product for the lips, measured according to the method described above, was equal to 5.7.

1. A cosmetic product for keratin materials comprising at least one first composition and at least one second composition, wherein the at least one first composition comprises a dispersion, in a liquid fatty phase, of particles of at least one graft ethylene polymer, and the at least one second composition, which is different from the at least one first composition, comprises a cosmetically acceptable medium.

2. The cosmetic product according to claim 1, wherein the at least one graft ethylene polymer comprises an ethylene backbone which is insoluble in the liquid fatty phase, and side chains which are covalently linked to the backbone and which are soluble in the liquid fatty phase.

3. The cosmetic product according to claim 1, wherein the at least one graft ethylene polymer is a graft acrylic polymer.

4. The cosmetic product according to claim 1, wherein the at least one graft ethylene polymer is dispersed in the absence of an additional stabilizer at the surface of the graft polymer particles.

5. The cosmetic product according to claim 1, wherein the at least one graft ethylene polymer in dispersion is an acrylic polymer, which can be obtained by free-radical polymerization in an organic medium for polymerization:
of at least one acrylic monomer and optionally of at least one additional nonacrylic vinyl monomer, to form the insoluble backbone; and
of at least one macromonomer comprising a polymerizable terminal group, to form the side chains, the macromonomer having a weight-average molecular mass greater than or equal to 200, and the polymerized macromonomer being present in an amount ranging from 0.05% to 20% by weight, relative to the total weight of the polymer.

6. The cosmetic product according to claim 1, wherein the at least one acrylic monomer is chosen from:
(i) the (meth)acrylates of formula: 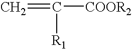
wherein:
R₁ is chosen from a hydrogen atom and a methyl group;
R₂ is a group chosen from:
linear and branched alkyl groups comprising from 1 to 6 carbon atoms, wherein the alkyl groups may comprise in their chain at least one heteroatom chosen from O, N and S; and/or may comprise at least one substituent chosen from —OH groups, halogen atoms, and —NR′R″ groups wherein R′ and R″, which may be identical or different, are chosen from linear and branched C₁-C₄ alkyls; and/or may be substituted with at least one polyoxyalkylene group, the polyoxyalkylene group comprising the repetition of 5 to 30 oxyalkylene units;
cyclic alkyl groups comprising from 3 to 6 carbon atoms, wherein the alkyl groups may comprise in their chain at least one heteroatom chosen from O, N and S, and/or may comprise at least one substituent chosen from OH groups and halogen atoms;
(ii) the (meth)acrylamides of formula: 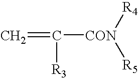
wherein:
R₃ is chosen from a hydrogen atom and a methyl group;
R₄ and R₅, which may be identical or different, are chosen from hydrogen atoms and linear and branched alkyl groups comprising from 1 to 6 carbon atoms, which may comprise at least one substituent chosen from —OH groups, halogen atoms, and —NR′R″ groups wherein R′ and R″, which may be identical or different, are chosen from linear and branched C₁-C₄ alkyls; or R₄ is a hydrogen atom and R₅ is a 1,1-dimethyl-3-oxobutyl group;
(iii) the (meth)acrylic monomers comprising at least one carboxylic, phosphoric or sulphonic acid functional group;
and the salts thereof.

7. The cosmetic product according to claim 5, wherein the at least one acrylic monomer is chosen from methyl, ethyl, propyl, butyl and isobutyl (meth)acrylates; methoxyethyl and ethoxyethyl (meth)acrylates; trifluoroethyl methacrylate; dimethylaminoethyl methacrylate, diethylaminoethyl methacrylate, 2-hydroxypropyl (meth)acrylate, 2-hydroxyethyl (meth)acrylate; dimethylaminopropylmethacrylamide; (meth)acrylic acid; and the salts thereof.

8. The cosmetic product according to claim 7, wherein the at least one acrylic monomer is chosen from methyl acrylate, methoxyethyl acrylate, methyl methacrylate, 2-hydroxyethyl methacrylate, (meth)acrylic acid, and dimethylaminoethyl methacrylate.

9. The cosmetic product according to claim 8, wherein the graft polymer comprises (meth)acrylic acid.

10. The cosmetic product according to claim 6, wherein the acrylic monomers comprise at least (meth)acrylic acid and at least one monomer chosen from the (meth)acrylates (i) and (meth)acrylamides (ii).

11. The cosmetic product according to claim 6, wherein the acrylic monomers comprise at least (meth)acrylic acid and at least one monomer chosen from C₁-C₃ alkyl (meth)acrylates.

12. The cosmetic product according to claim 9, wherein the (meth)acrylic acid is present in an amount greater than or equal to 5% by weight, relative to the total weight of the polymer.

13. The cosmetic product according to claim 12, wherein the (meth)acrylic acid is present in an amount greater than or equal to 15% by weight, relative to the total weight of the polymer.

14. The cosmetic product according to claim 12, wherein the (meth)acrylic acid is present in an amount ranging from 5% to 80% by weight, relative to the total weight of the polymer.

15. The cosmetic product according to claim 13, wherein the (meth)acrylic acid is present in an amount ranging from 15% to 60% by weight, relative to the total weight of the polymer.

16. The cosmetic product according to claim 5, wherein the at least one graft acrylic polymer does not comprise an additional nonacrylic vinyl monomer.

17. The cosmetic product according to claim 5, wherein the at least one graft acrylic polymer can be obtained by free-radical polymerization of at least one acrylic monomer and of at least one additional nonacrylic vinyl monomer, and of the macromonomer.

18. The cosmetic product according to claim 17, wherein the at least one additional nonacrylic vinyl monomer is chosen from:
the vinyl esters of formula: R₆—COO—CH═CH₂
wherein R₆ is chosen from linear and branched alkyl groups comprising from 1 to 6 atoms, and cyclic alkyl groups comprising from 3 to 6 carbon atoms and/or an aromatic group;
the nonacrylic vinyl monomers comprising at least one group chosen from carboxylic, phosphoric and sulphonic acid functional groups, and the salts thereof; and
the nonacrylic vinyl monomers comprising at least one tertiary amine functional group.

19. The cosmetic product according to claim 5, wherein the at least one acrylic monomer is present in an amount ranging from 50% to 100% by weight, relative to the weight of the acrylic monomer and optional nonacrylic vinyl monomer mixture.

20. The cosmetic product according to claim 19, wherein the at least one acrylic monomer is present in an amount ranging from 70% to 100% by weight, relative to the weight of the acrylic monomer and optional nonacrylic vinyl monomer mixture.

21. The cosmetic product according to claim 5, wherein the macromonomer comprises at one of the chain ends a polymerizable end group chosen from a vinyl group or a (meth)acrylate group.

22. The cosmetic product according to claim 5, wherein the macromonomer has a weight-average molecular mass greater than or equal to 300.

23. The cosmetic product according to claim 22, wherein the macromonomer has a weight-average molecular mass greater than or equal to 600.

24. The cosmetic product according to claim 5, wherein the macromonomer has a weight-average molecular mass (Mw) ranging from 200 to 100,000.

25. The cosmetic product according to claim 24, wherein the macromonomer has a weight-average molecular mass (Mw) ranging from 800 to 6,000.

26. The cosmetic product according to claim 5, wherein the polymerized macromonomer is present in an amount ranging from 0.1% to 15% by weight, relative to the total weight of the polymer.

27. The cosmetic product according to claim 26, wherein the polymerized macromonomer is present in an amount ranging from 0.3 to 8% by weight, relative to the total weight of the polymer.

28. The cosmetic product according to claim 1, wherein the liquid fatty phase comprises at least one organic liquid compound chosen from:
organic liquid compounds having an overall solubility parameter according to the Hansen solubility space of less than or equal to 18 (MPa)^{1/2}, and
monoalcohols having an overall solubility parameter according to the Hansen solubility space of less than or equal to 20 (MPa)^{1/2}.

29. The cosmetic product according to claim 1, wherein the liquid fatty phase comprises at least one volatile oil.

30. The cosmetic product according to claim 1, wherein the liquid fatty phase is a nonsilicone liquid fatty phase.

31. The cosmetic product according to claim 30, wherein the nonsilicone liquid fatty phase comprises, in an amount greater than or equal to 50% by weight, at least one nonsilicone organic liquid compound chosen from:
organic liquid nonsilicone compounds having an overall solubility parameter according to the Hansen solubility space of less than or equal to 18 (MPa)^{1/2}; and
liquid monoalcohols having an overall solubility parameter according to the Hansen solubility space of less than or equal to 20 (MPa)^{1/2}.

32. The cosmetic product according to claim 30, wherein the nonsilicone liquid fatty phase comprises less than 50% by weight, relative to the weight of the nonsilicone liquid fatty phase, of the at least one silicone organic liquid compound having an overall solubility parameter according to the Hansen solubility space of less than or equal to 18 (MPa)^{1/2}.

33. The cosmetic product according to claim 30, wherein the nonsilicone liquid fatty phase does not comprise silicone organic liquid compounds.

34. The cosmetic product according to claim 30, wherein the macromonomer is a carbonaceous macromonomer.

35. The cosmetic product according to claim 34, wherein the carbonaceous macromonomer is chosen from:
(i) linear and branched C₈-C₂₂ alkyl acrylate and methacrylate homopolymers, and copolymers having a polymerizable end group chosen from vinyl and (meth)acrylate groups; and
(ii) olefins having a polymerizable end group with ethylenic unsaturation.

36. The cosmetic product according to claim 34, wherein the carbonaceous macromonomer is chosen from:
(i) macromonomers of poly(2-ethylhexyl acrylate) with a mono(meth)acrylate end; macromonomers of poly(dodecyl acrylate) with a mono(meth)acrylate end; macromonomers of poly(dodecyl methacrylate); macromonomers of poly(stearyl acrylate) with a mono(meth)acrylate end; macromonomers of poly(stearyl methacrylate) with a mono(meth)acrylate end; and
(ii) polyethylene macromonomers, polypropylene macromonomers, polyethylene/polypropylene copolymer macromonomers, polyethylene/polybutylene copolymer macromonomers, polyisobutylene macromonomers; polybutadiene macromonomers; polyisoprene macromonomers; polybutadiene macromonomers, and poly(ethylene/butylene)polyisoprene macromonomers, these macromonomers having a (meth)acrylate end group.

37. The cosmetic product according to claim 34, wherein the carbonaceous macromonomer is chosen from:
(i) macromonomers of poly(2-ethylhexyl acrylate) with a mono(meth)acrylate end, macromonomers of poly(dodecyl acrylate) with a mono(meth)acrylate end; and
(ii) poly(ethylene/butylene) methacrylate.

38. The cosmetic product according to claim 34, wherein the at least one graft polymer is chosen from the polymers obtained via polymerization:
of methyl acrylate and the polyethylene/polybutylene macromonomer with a methacrylate end group;
of methoxyethyl acrylate and the polyethylene/polybutylene macromonomer with a methacrylate end group;
of the methyl acrylate/methyl methacrylate monomers and the polyethylene/polybutylene macromonomer with a methacrylate end group;
of the methyl acrylate/acrylic acid monomers and the polyethylene/polybutylene macromonomer with a methacrylate end group;
of the methyl acrylate/dimethylaminoethyl methacrylate monomers and the polyethylene/polybutylene macromonomer with a methacrylate end group; and
of the methyl acrylate/2-hydroxyethyl methacrylate monomers and the polyethylene/polybutylene macromonomer with a methacrylate end group.

39. The cosmetic product according to claim 30, wherein the at least one graft polymer is a nonsilicone graft polymer.

40. The cosmetic product according to claim 39, wherein the nonsilicone graft polymer comprises a carbonaceous macromonomer and optionally comprises a silicone macromonomer, wherein the silicone macromonomer, if present, is present in an amount less than or equal to 7% by weight, relative to the total weight of the polymer.

41. The cosmetic product according to claim 39, wherein the nonsilicone graft polymer is free of silicone macromonomer.

42. The cosmetic product according to claim 1, wherein the liquid fatty phase is a silicone liquid fatty phase.

43. The cosmetic product according to claim 42, wherein the silicone liquid fatty phase comprises at least one silicone organic liquid compound, present in an amount greater than or equal to 50% by weight, chosen from silicone organic liquid compounds having an overall solubility parameter according to the Hansen solubility space of less than or equal to 18 (MPa)^{1/2}.

44. The cosmetic product according to claim 32, wherein the silicone organic liquid compound comprises at least one volatile silicone oil.

45. the cosmetic product according to claim 44, wherein the at least one volatile silicone oil is chosen from octamethylcyclotetrasiloxane, decamethylcyclopentasiloxane, dodecamethylcyclohexasiloxane, heptamethylhexyltrisiloxane, heptamethyloctyltrisiloxane, octamethyltrisiloxane, and decamethyltetrasiloxane.

46. The cosmetic product according to claim 32, wherein the silicone organic liquid compound comprises at least one non-volatile silicone oil.

47. The cosmetic product according to claim 46, wherein the at least one non-volatile silicone oil is chosen from non-volatile polydialkylsiloxanes; polydimethylsiloxanes comprising alkyl, alkoxy or phenyl groups, which are pendant or at the silicone chain end, groups comprising from 2 to 24 carbon atoms; phenylated silicones; polysiloxanes modified with fatty acids, fatty alcohols, polyoxyalkylenes; aminated polysiloxanes; polysiloxanes with hydroxyl groups; and fluorinated polysiloxanes comprising a fluorinated group which is pendant or at the silicone chain end comprising from 1 to 12 carbon atoms in which all or some of the hydrogens are substituted with fluorine atoms.

48. The cosmetic product according to claim 42, wherein the liquid fatty phase comprises less than 50% by weight of nonsilicone organic liquid compounds.

49. The cosmetic product according to claim 31, wherein the at least one nonsilicone organic liquid compound is chosen from nonsilicone organic liquid compounds having an overall solubility parameter according to the Hansen solubility space of less than 18 (MPa)^{1/2}; and liquid monoalcohols having an overall solubility parameter according to the Hansen solubility space of less than or equal to 20 (MPa)^{1/2}.

50. The cosmetic product according to claim 31, wherein the at least one nonsilicone organic liquid compound having an overall solubility parameter according to the Hansen solubility space of less than or equal to 18 (MPa)^{1/2} is chosen from carbonaceous, hydrocarbonaceous or fluorinated oils alone or as a mixture; optionally volatile linear, branched and/or cyclic alkanes; esters; ketones; and ethers.

51. The cosmetic product according to claim 31, wherein the monoalcohols having an overall solubility parameter according to the Hansen solubility space of less than or equal to 20 (MPa)^{1/2} are chosen from aliphatic fatty monoalcohols comprising from 6 to 30 carbon atoms, and the hydrocarbon chain containing no substituent group.

52. The cosmetic product according to claim 29, wherein the at least one volatile oil is chosen from nonsilicone volatile oils.

53. The cosmetic product according to claim 52, wherein the at least one nonsilicone volatile oil is chosen from isododecane, isodecane and isohexadecane.

54. The cosmetic product according to claim 42, wherein the liquid fatty phase does not comprise any nonsilicone organic liquid compounds.

55. The cosmetic product according to claim 42, wherein the macromonomer is chosen from silicone macromonomers.

56. The cosmetic product according to claim 55, wherein the silicone macromonomer is chosen from organopolysiloxane macromonomers.

57. The cosmetic product according to claim 55, wherein the macromonomer is chosen from polydimethylsiloxanes with a mono(meth)acrylate end group.

58. The cosmetic product according to claim 55, wherein the at least one silicone macromonomer is chosen from macromonomers of formula (II): 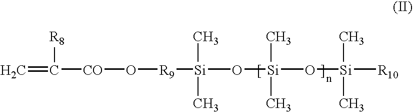
wherein R₈ is chosen from a hydrogen atom and a methyl group; R₉ is chosen from divalent hydrocarbon groups comprising from 1 to 10 carbon atoms and optionally comprises one or two ether bonds —O—; R₁₀ is chosen from alkyl groups having from 1 to 10 carbon atoms; and n is an integer ranging from 1 to 300.

59. The cosmetic product according to claim 6, wherein the at least one graft acrylic polymer can be obtained via free-radical polymerization in the polymerization medium:
of a principal acrylic monomer chosen from C₁-C₃ alkyl (meth)acrylates, alone or as a mixture, and optionally of at least one additional acrylic monomers chosen from acrylic acid, methacrylic acid and the alkyl (meth)acrylates of formula (I), and the salts thereof, to form the insoluble backbone: 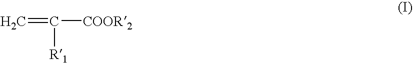
wherein:
R′₁ is chosen from a hydrogen atom and methyl groups;
R′₂ is chosen from:
linear and branched alkyl groups comprising from 1 to 6 carbon atoms, the group comprising in its chain at least one oxygen atom and/or comprising at least one substituent chosen from —OH groups, halogen atoms, and —NR′R″ groups wherein R′ and R″, which may be identical or different, are chosen from linear and branched C₁-C₃ alkyls;
cyclic alkyl groups comprising from 3 to 6 carbon atoms, wherein the cyclic alkyl groups may comprise in their chain at least one oxygen atom and/or may comprise at least one substituent chosen from OH groups and halogen atoms;
and of a silicone macromonomer.

60. The cosmetic product according to claim 59, wherein R′₂ is a group chosen from methoxyethyl, ethoxyethyl, trifluoroethyl, 2-hydroxyethyl, 2-hydroxypropyl, dimethylaminoethyl, diethylaminoethyl, and dimethylaminopropyl groups.

61. The cosmetic product according to claim 59, wherein the principal acrylic monomer is chosen from methyl (meth)acrylate, ethyl (meth)acrylate, n-propyl (meth)acrylate, isopropyl (meth)acrylate and mixtures thereof.

62. The cosmetic product according to claim 61, wherein the principal acrylic monomer is chosen from methyl acrylate, methyl methacrylate and ethyl methacrylate.

63. The cosmetic product according to claim 59, wherein the at least one additional acrylic monomer is chosen from (meth)acrylic acid, methoxyethyl (meth)acrylate, ethoxyethyl (meth)acrylate, trifluoroethyl methacrylate, dimethylaminoethyl methacrylate, diethylaminoethyl methacrylate, 2-hydroxypropyl (meth)acrylate, 2-hydroxyethyl (meth)acrylate, and the salts thereof.

64. The cosmetic product according to claim 63, wherein the at least one additional acrylic monomer is chosen from acrylic acid and methacrylic acid.

65. The cosmetic product according to claim 1, wherein the at least one graft polymer is chosen from the polymers obtained via polymerization:
of methyl acrylate and the monomethacryloxypropylpolydimethylsiloxane macromonomer having a weight-average molecular weight ranging from 800 to 6,000; and
of methyl acrylate, acrylic acid and the monomethacryloxypropylpolydimethylsiloxane macromonomer having a weight-average molecular weight ranging from 800 to 6,000.

66. The cosmetic product according to claim 1, wherein the at least one graft polymer is a silicone graft polymer.

67. The cosmetic product according to claim 66, wherein the at least one silicone graft polymer comprises a silicone macromonomer and optionally comprises a carbonaceous macromonomer, in an amount less that or equal to 7% by weight, relative to the total weight of the polymer.

68. The cosmetic product according to claim 67, wherein the at least one silicone graft polymer does not comprise a carbonaceous macromonomer.

69. The cosmetic product according to claim 1, wherein the at least one graft polymer has a weight-average molecular mass (Mw) ranging from 10,000 to 300,000.

70. The cosmetic product according to claim 69, wherein the at least one graft polymer has a weight-average molecular mass (Mw) ranging from 25,000 to 150,000.

71. The cosmetic product according to claim 1, wherein the at least one graft polymer particles have a mean size ranging from 10 nm to 400 nm.

72. The cosmetic product according to claim 71, wherein the at least one graft polymer particles have a mean size ranging from 20 nm to 200 nm.

73. The cosmetic product according to claim 1, wherein the at least one graft ethylene polymer is a film-forming polymer.

74. The cosmetic product according to claim 1, wherein the at least one graft polymer is present in an amount ranging from 0.1% to 70% by weight, relative to the total weight of the composition

75. The cosmetic product according to claim 74, wherein the at least one graft polymer is present in an amount ranging from 1% to 40% by weight, relative to the total weight of the composition.

76. The cosmetic product according to claim 1, wherein at least one of the at least one first composition and at least one second composition is in the form of a paste or a stick.

77. The cosmetic product according to claim 1, wherein the at least one second composition is transparent.

78. The cosmetic product according to claim 1, wherein the at least one second composition comprises less than 5% by weight of pigments.

79. The cosmetic product according to claim 78, wherein the at least one second composition comprises less than 1% by weight of pigments.

80. The cosmetic product according to claim 1, wherein the transfer index of the cosmetic product is less than or equal to 40 out of 100.

81. The cosmetic product according to claim 80, wherein the transfer index of the cosmetic product is less than or equal to 20 out of 100.

82. The cosmetic product according to claim 81, wherein the transfer index of the cosmetic product is less than or equal to 2 out of 100.

83. The cosmetic product according to claim 1, wherein the mean gloss of the cosmetic product, measured at 20° C., is greater than or equal to 30.

84. The cosmetic product according to claim 83, wherein the mean gloss of the cosmetic product, measured at 20° C., is greater than or equal to 60.

85. The cosmetic product according to claim 1, wherein the staying power index of the cosmetic product is greater than or equal to 80%.

86. The cosmetic product according to claim 85, wherein the staying power index of the cosmetic product is greater than or equal to 95%.

87. The cosmetic product according to claim 1, wherein the cosmetically acceptable medium of the at least one second composition comprises at least one silicone polymer chosen from dimethiconols, and polydimethylsiloxanes.

88. The cosmetic product according to claim 1, wherein the at least one first composition and/or the at least one second composition comprises at least one coloring agent chosen from fat-soluble colorants, water-soluble colorants, pigments, and pearlescent agents.

89. The cosmetic product according to claim 1, wherein the at least one first composition and/or the at least one second composition is in the form of an anhydrous liquid, an anhydrous stick or an emulsion.

90. The cosmetic product according to claim 1, wherein the cosmetic product is in the form of a foundation, a blusher or eyeshadow, a lipstick, a product having care properties; a mascara, an eye liner, a nail varnish, a concealer product or a make-up product for the body.

91. A method for making up keratin materials, comprising
applying to the keratin material a first coat of at least one first composition comprising a dispersion, in a liquid fatty phase, of particles of at least one graft ethylene polymer and at least one coloring agent,
and then applying, over all or part of the first coat, a second coat of at least one second composition comprising a cosmetically acceptable medium,
wherein the keratin material is chosen from the skin and/or the lips and/or the superficial body growths, and
wherein the at least one first composition and at least one second composition are present in an effective amount to confer on the skin and/or the lips and/or the superficial body growths a make-up with at least one property chosen from comfortable, glossy, transfer-resistant, non-migrating, good color staying power, and good gloss staying power.

92. The method according to claim 91, wherein the at least one second composition is chosen so as to improve at least one cosmetic property of the at least one first composition, compared to when the at least one first composition is applied alone to the keratin material.

93. The method according to claim 91, additionally comprising at least partially drying the at least one first composition before applying the coat of the at least one second composition.

94. A make-up kit comprising
at least one first compartment comprising at least one first composition comprising a dispersion, in a liquid fatty phase, of particles of at least one graft ethylene polymer,
and at least one second compartment comprising at least one second composition comprising a cosmetically acceptable medium.

95. The make-up kit according to claim 94, wherein the at least one first compartment and the at least one second compartment are formed inside the same container.

96. The make-up kit according to claim 94, wherein the at least one first compartment and the at least one second compartment are formed in two separate containers.
